(19) United States
(12) Patent Application Publication
(10) Pub. No.: US 2026/0242477 A1
(43) Pub. Date: Aug. 20, 2026

(54) COMBINATION OF PD-1 INHIBITORS AND LAG-3 INHIBITORS FOR ENHANCED EFFICACY IN TREATING MELANOMA

(71) Applicant: Regeneron Pharmaceuticals, Inc., Tarrytown, NY (US)

(72) Inventors: Karl D. Lewis, Eastchester, NY (US); Matthew Fury, Katonah, NY (US); Mark Salvati, Bronxville, NY (US); Israel Lowy, Dobbs Ferry, NY (US)

(21) Appl. No.: 19/162,290

(22) PCT Filed: Mar. 12, 2024
(86) PCT No.: PCT/US2024/019579

§ 371 Date: Sep. 4, 2025

Related U.S. Application Data
(60) Provisional application No. 63/489,900, filed on Mar. 13, 2023.

(51) Int. Cl. C07K 16/28; A61K 39/00; A61K 45/06; A61P 35/04

(52) U.S. Cl. CPC C07K 16/2818; A61K 45/06; A61P 35/04; C07K 16/2803; A61K 2039/507; A61K 2039/545; C07K 2317/565

(57) ABSTRACT

The present disclosure provides methods for treating or inhibiting the growth of melanoma comprising selecting a patient with melanoma and administering a LAG-3 inhibitor in combination with a PD-1 inhibitor (e.g., an anti-PD-1 antibody or antigen-binding fragment thereof). In certain embodiments, the administration of the PD-1 inhibitor enhances the efficacy of a LAG-3 inhibitor (e.g., an anti-LAG-3 antibody or antigen-binding fragment thereof) in inhibiting the growth of melanoma.

### CROSS-REFERENCE TO RELATED APPLICATIONS

This application claims priority to U.S. Provisional Patent Application No. 63/489,900, filed Mar. 13, 2023, the contents of which are incorporated by reference herein.

### TECHNICAL FIELD

The present disclosure provides, in part, compositions, including inhibitors of LAG-3 and PD-1, and methods for treating melanoma.

### SEQUENCE LISTING

An official copy of the sequence listing is submitted concurrently with the specification electronically via Patent Center. The contents of the electronic sequence listing (11445WO01_Sequence_Listing_ST26.xml; Size: 28,672 bytes; and Date of Creation: Mar. 12, 2024) is herein incorporated by reference in its entirety.

### BACKGROUND

Programmed death-1 (PD-1) receptor signaling in the tumor microenvironment plays a key role in allowing tumor cells to escape immune surveillance by the host immune system. The PD-1 receptor has two ligands, PD-ligand-1 (PD-L1) and PD-L2. Blockade of the PD-1 signaling pathway has demonstrated clinical activity in patients with multiple tumor types, and antibody therapeutics that block PD-1/PDL1 signaling (e.g., nivolumab, pembrolizumab, atezolizumab, durvalumab, and cemiplimab) have been approved for the treatment of various cancers including, for example, metastatic melanoma and metastatic squamous non-small cell lung cancer.

Like PD-1, lymphocyte activation gene-3 (LAG-3) negatively regulates T-cell activity. LAG-3 (also called CD223) is a 503 amino acid transmembrane protein receptor expressed on activated CD4 and CD8 T cells, γδ T cells, natural killer T cells, B-cells, natural killer cells, plasmacytoid dendritic cells and regulatory T cells. LAG-3 is a member of the immunoglobulin (Ig) superfamily. The primary function of LAG-3 is to attenuate the immune response. LAG-3 binding to MHC class II molecules results in delivery of a negative signal to LAG-3-expressing cells and down-regulates antigen-dependent CD4 and CD8 T cell responses. LAG-3 negatively regulates the ability of T cells to proliferate, produce cytokines and lyse target cells, termed as ‘exhaustion’ of T cells. LAG-3 is also reported to play a role in enhancing T regulatory (Treg) cell function (Pardoll 2012, Nature Reviews Cancer 12: 252-264).

Since both PD-1 and LAG-3 play important roles in tumor immunity, they are ideal targets for immunotherapy. Targeting both LAG-3 and PD-1 (including in anti-PD-1 resistant tumors) may result in objective responses in patients across several tumor types.

### SUMMARY

The present disclosure relates to methods for treating melanoma and methods for inhibiting tumor growth.

Provided herein are methods for treating, ameliorating at least one symptom or indication, or inhibiting the growth of melanoma in a subject. The methods according to this aspect of the disclosure comprise administering a therapeutically effective amount of an antibody or antigen-binding fragment thereof that specifically binds to programmed death 1 (PD-1) in combination with a therapeutically effective amount of an antibody or antigen-binding fragment thereof that specifically binds to LAG-3 to a subject in need thereof. In the methods disclosed herein, the inhibition achieved with the combination therapy is more efficacious than administration of either antibody as a monotherapy.

Provided herein are methods of treating melanoma or inhibiting the growth of a melanoma. In some embodiments, the method comprises administering to a subject in need thereof a therapeutically effective amount each of (a) an antibody or antigen-binding fragment thereof that specifically binds programmed death 1 (PD-1); and (b) an antibody or antigen-binding fragment thereof that specifically binds lymphocyte activation gene-3 (LAG-3). In some aspects, the melanoma is unresectable locally advanced melanoma, unresectable metastatic melanoma, or completely resected high-risk melanoma.

A therapeutically effective amount can be one or more doses of the antibody or antigen-binding fragment thereof. In some aspects, one dose of the anti-PD-1 antibody or antigen-binding fragment thereof comprises 50 to 1500 mg. In some aspects, one dose of the anti-PD-1 antibody or antigen-binding fragment thereof comprises 350 mg. In some aspects, one dose of the anti-LAG-3 antibody or antigen-binding fragment thereof comprises between 50 to 8000 mg. In some aspects, one dose of the anti-LAG-3 antibody or antigen-binding fragment thereof comprises 400 mg. In some aspects, one dose of the anti-LAG-3 antibody or antigen-binding fragment thereof comprises 1600 mg.

The anti-LAG-3 antibody or antigen-binding fragment thereof can be administered prior to, concurrent with or after the anti-PD-1 antibody or antigen-binding fragment thereof. In some aspects, the anti-LAG-3 antibody or antigen-binding fragment thereof is administered prior to the anti-PD-1 antibody or antigen-binding fragment thereof. In some aspects, the anti-LAG-3 antibody or antigen-binding fragment thereof is administered the same day as the anti-PD-1 antibody or antigen-binding fragment thereof.

In some embodiments, two or more doses of the anti-LAG-3 antibody or antigen-binding fragment thereof are administered in combination with two or more doses of the anti-PD-1 antibody or antigen-binding fragment thereof. In some aspects, each dose of the anti-PD-1 antibody or antigen-binding fragment thereof comprises 350 mg. In some aspects, each dose of the anti-LAG-3 antibody or antigen-binding fragment thereof comprises between 50 and 8000 mg. In some aspects, each dose of the anti-LAG-3 antibody or antigen-binding fragment thereof comprises 1600 mg. In some aspects, each dose of the anti-LAG-3 antibody or antigen-binding fragment thereof comprises 400 mg. In some aspects, each dose of the anti-PD-1 antibody or antigen-binding fragment thereof comprises 200 mg, 250 mg or 350 mg and each dose of the anti-LAG-3 antibody or antigen-binding fragment thereof comprises 400 mg, 800 mg, 1000 mg, 1400 mg, or 1600 mg. The antibodies, e.g., the dosages comprising the antibodies, can be administered intravenously, subcutaneously, or intraperitoneally.

When two or more doses of the anti-LAG-3 antibody or antigen-binding fragment thereof are administered in combination with two or more doses of the anti-PD-1 antibody or antigen-binding fragment thereof, each dose of the anti-PD-1 antibody or antigen-binding fragment thereof can be administered 0.5 weeks to 12 weeks after the immediately preceding dose. In some aspects, each dose of the anti-LAG-3 antibody or antigen-binding fragment thereof is administered 0.5 weeks to 12 weeks after the immediately preceding dose. In some aspects, each dose of the anti-PD-1 antibody or antigen-binding fragment thereof is administered once in six weeks. In some aspects, each dose of the anti-LAG-3 antibody or antigen-binding fragment thereof is administered once in six weeks. In some aspects, each dose of the anti-PD-1 antibody or antigen-binding fragment thereof is administered once in three weeks. In some aspects, each dose of the anti-LAG-3 antibody or antigen-binding fragment thereof is administered once in three weeks.

In some embodiments, the melanoma is unresectable locally advanced melanoma. In some aspects, the melanoma is unresectable metastatic melanoma. In such embodiments, the patient can be further selected as having one or more of the following criteria: (i) at least 12 years of age on the date of providing informed consent; (ii) either stage IIC, III, or stage IV per AJCC 8th edition (Amin, 2017) and have histologically confirmed melanoma that is completely surgically resected in order to be eligible; (iii) patients with stage IIIA disease must have at least 1 lymph node micrometastasis measuring >1 mm in greatest diameter; (iv) Stage IIC melanoma confirmed by a pathologically negative Sentinel lymph node biopsy (SLNB) specimen and no evidence of regional or distant metastasis; (v) complete surgical resection performed within 12 weeks prior to treatment, and treatment may occur only after satisfactory wound healing from the surgery; (vi) disease-free status as documented by a complete physical examination and imaging studies prior to treatment; (vii) patient shows ≥1% LAG3 in the tumor tissue as determined by IHC or iPET; and (viii) patients must not have received systemic anti-cancer therapy or radiation therapy for melanoma in the previous 5 years.

In some embodiments, the melanoma is completely resected high-risk melanoma. In such embodiments, the patient is further selected as having one or more of the following criteria: (1) ≥12 years of age; (2) Stage IIc, III or IV (all M-stages) and histologically confirmed melanoma, completely resected <12 weeks prior to treatment; (3) no prior systemic anti-cancer therapy or radiation therapy for melanoma in the previous 5 years; (4) shows ≥1% LAG3 in the tumor tissue as determined by IHC or iPET; (5) no evidence of metastatic disease; and (6) an Eastern Cooperative Oncology Group performance status (PS) of 0 or 1 (for adult pts), Karnofsky PS>70 (pts>16 years) or Lansky PS>70 (pts<16 years).

Provided herein are methods of treating melanoma or inhibiting the growth of melanoma. In some embodiments, the method comprises (1) selecting a patient with melanoma, wherein the patient has unresectable locally advanced melanoma, unresectable metastatic melanoma, or completely resected high-risk melanoma; and (2) administering to the patient (a) 350 mg anti-PD-1 antibody or antigen-binding fragment thereof comprising the HCVR/LCVR amino acid sequence pair of SEQ ID NOs: 1/2; and (b) 400 mg or 1600 mg anti-LAG-3 antibody or antigen-binding fragment thereof comprising the HCVR/LCVR amino acid sequence pair of SEQ ID NOs: 11/12.

In some aspects, the patient is further selected as having one or more of the following criteria: (1) ≥12 years of age; (2) Stage IIc, III or IV (all M-stages) and histologically confirmed melanoma, completely resected <12 weeks prior to treatment; (3) no prior systemic anti-cancer therapy or radiation therapy for melanoma in the previous 5 years; (4) no evidence of metastatic disease on staging investigations; and (5) an Eastern Cooperative Oncology Group performance status (PS) of 0 or 1 (for adult pts), Karnofsky PS>70 (pts>16 years) or Lansky PS>70 (pts<16 years).

In some aspects, the administering of step (2) occurs once every 3 weeks. In some aspects, the administering of step (2) occurs once every 6 weeks.

Provided herein are methods of treating melanoma or inhibiting the growth of melanoma comprising: (1) selecting a patient with an unresectable locally advanced melanoma, unresectable metastatic melanoma, or completely resected high-risk melanoma, wherein the selected patient has not received prior systemic treatment for advanced disease; and (2) administering to the patient (a) 350 mg anti-PD-1 antibody or antigen-binding fragment thereof comprising the HCVR/LCVR amino acid sequence pair of SEQ ID NOs: 1/2; and (b) 400 mg or 1600 mg anti-LAG-3 antibody or antigen-binding fragment thereof comprising the HCVR/LCVR amino acid sequence pair of SEQ ID NOs: 11/12.

In some aspects, the administering of step (2) occurs once every 3 weeks. In some aspects, the administering of step (2) once every 6 weeks.

In some aspects, patient is further selected as having one or more of the following criteria: (1) ≥12 years of age; (2) Stage IIc, III or IV (all M-stages) and histologically confirmed melanoma, completely resected <12 weeks prior to treatment; (3) no prior systemic anti-cancer therapy or radiation therapy for melanoma in the previous 5 years; (4) no evidence of metastatic disease on staging investigations; and (5) an Eastern Cooperative Oncology Group performance status (PS) of 0 or 1 (for adult pts), Karnofsky PS>70 (pts>16 years) or Lansky PS>70 (pts<16 years).

Provided herein are methods of treating melanoma or inhibiting the growth of melanoma comprising: (1) selecting a patient with a melanoma; and (2) administering to the patient (a) 400 mg or 1600 mg anti-LAG-3 antibody or antigen-binding fragment thereof comprising the HCVR/LCVR amino acid sequence pair of SEQ ID NOs: 11/12 in combination with (a), (b) 350 mg anti-PD-1 antibody or antigen-binding fragment thereof comprising the HCVR/LCVR amino acid sequence pair of SEQ ID NOs: 1/2.

The step of administering can occur once every 3 weeks. In some aspects, the step of administering occurs once every 6 weeks.

In some aspects, the treatment produces a therapeutic effect selected from the group consisting of delay in melanoma growth, reduction in melanoma cell number, melanoma regression, increase in survival, partial response, and complete response. In some aspects, the melanoma growth can be delayed by at least 10 days as compared to an untreated subject. In some aspects, the melanoma growth is inhibited by at least 50% as compared to an untreated subject. In some aspects, the melanoma growth is inhibited by at least 20% as compared to a subject administered with either antibody as monotherapy.

In some embodiments, the methods further comprise administering to the subject an additional therapeutic agent or therapy, wherein the additional therapeutic agent or therapy is selected from the group consisting of radiation, surgery, a chemotherapeutic agent, a cancer vaccine, a PD-L1 inhibitor, a CTLA-4 inhibitor, a TIM3 inhibitor, a BTLA inhibitor, a TIGIT inhibitor, a CD47 inhibitor, a CD28 agonist, a CD38 inhibitor, an indoleamine-2,3-dioxygenase (IDO) inhibitor, a vascular endothelial growth factor (VEGF) antagonist, an angiopoietin-2 (Ang2) inhibitor, a transforming growth factor beta (TGFβ) inhibitor, an epidermal growth factor receptor (EGFR) inhibitor, an antibody to a tumor-specific antigen, Bacillus Calmette-Guerin vaccine, granulocyte-macrophage colony-stimulating factor, an oncolytic virus, a cytotoxin, an interleukin 6 receptor (IL-6R) inhibitor, an interleukin 4 receptor (IL-4R) inhibitor, an IL-10 inhibitor, IL-2, IL-7, IL-21, IL-12, IL-15, an antibody-drug conjugate, a GITR agonist, a 4-1BB agonist, CD20xCD3 bispecific antibody, MUC16xCD3 bispecific antibody, and an anti-inflammatory drug.

In some embodiments, the anti-PD-1 antibody or antigen-binding fragment thereof comprises the heavy chain complementarity determining regions (HCDR1, HCDR2 and HCDR3) of a heavy chain variable region (HCVR) and three light chain complementarity determining regions (LCDR1, LCDR2 and LCDR3) of a light chain variable region (LCVR), wherein HCDR1 comprises the amino acid sequence of SEQ ID NO: 3; HCDR2 comprises the amino acid sequence of SEQ ID NO: 4; HCDR3 comprises the amino acid sequence of SEQ ID NO: 5; LCDR1 comprises the amino acid sequence of SEQ ID NO: 6; LCDR2 comprises the amino acid sequence of SEQ ID NO: 7; and LCDR3 comprises the amino acid sequence of SEQ ID NO: 8. In some aspects, the HCVR comprises the amino acid sequence of SEQ ID NO: 1 and the LCVR comprises the amino acid sequence of SEQ ID NO: 2. In some aspects, the anti-PD-1 antibody comprises a heavy chain comprising the amino acid sequence of SEQ ID NO: 9 and a light chain comprising the amino acid sequence of SEQ ID NO: 10.

In some embodiments, the anti-LAG-3 antibody or antigen-binding fragment thereof comprises the heavy chain CDRs (HCDR1, HCDR2 and HCDR3) of a HCVR and three light chain CDRs (LCDR1, LCDR2 and LCDR3) of a LCVR, wherein HCDR1 comprises the amino acid sequence of SEQ ID NO: 13; HCDR2 comprises the amino acid sequence of SEQ ID NO: 14; HCDR3 comprises the amino acid sequence of SEQ ID NO: 15; LCDR1 comprises the amino acid sequence of SEQ ID NO: 16; LCDR2 comprises the amino acid sequence of SEQ ID NO: 17; and LCDR3 comprises the amino acid sequence of SEQ ID NO: 18. In some aspects, the HCVR comprises the amino acid sequence of SEQ ID NO: 11 and the LCVR comprises the amino acid sequence of SEQ ID NO: 12. In some aspects, the anti-LAG-3 antibody comprises a heavy chain comprising the amino acid sequence of SEQ ID NO: 19 and a light chain comprising the amino acid sequence of SEQ ID NO: 20.

Provided herein are methods of treating melanoma or inhibiting the growth of melanoma. In some aspects, the methods comprise: (a) selecting a patient with melanoma wherein the patient has completed surgery to treat the melanoma; and (b) administering to the patient: (1) an initial loading dose comprising an anti-PD-1 antibody or antigen-binding fragment thereof comprising the HCVR/LCVR amino acid sequence pair of SEQ ID NOs: 1/2; and an anti-LAG-3 antibody or antigen-binding fragment thereof comprising the HCVR/LCVR amino acid sequence pair of SEQ ID NOs: 11/12; and (2) one or more secondary doses, wherein the one or more secondary doses occur one to four weeks after the immediately preceding dose. In some aspects, the one or more secondary doses occur three weeks after the immediately preceding dose.

In some aspects, the patient shows≥1% of more LAG3 in the melanoma tissue. In some aspects, the patient is diagnosed with Stage IV melanoma.

In some aspects, the patient is further selected as having one or more of the following criteria: (1) ≥12 years of age; (2) Stage IIc, III or IV (all M-stages) and histologically confirmed melanoma, completely resected <12 weeks prior to treatment; (3) no prior systemic anti-cancer therapy or radiation therapy for melanoma in the previous 5 years; (4) shows 21% LAG3 in the tumor tissue as determined by IHC or iPET; (5) no evidence of metastatic disease; and (6) an Eastern Cooperative Oncology Group performance status (PS) of 0 or 1 (for adult pts), Karnofsky PS>70 (pts>16 years) or Lansky PS>70 (pts<16 years).

In some aspects, the methods comprise further administering to a patient in need thereof: (3) one or more tertiary doses, wherein the one or more tertiary doses occur three to twelve weeks after the immediately preceding dose. In some aspects, the one or more tertiary doses occur three weeks or six weeks after the immediately preceding dose.

In some aspects, the initial loading dose comprises (a) 50 mg to 1500 mg anti-PD-1 antibody or antigen-binding fragment thereof and (b) 50 mg to 8000 mg anti-LAG-3 antibody or antigen-binding fragment thereof. In some aspects, the one or more secondary doses comprise: (a) 350 mg anti-PD-1 antibody or antigen-binding fragment thereof and (b) 400 mg, 800 mg, 1000 mg, 1400 mg, 1600 mg or 2000 mg anti-LAG-3 antibody or antigen-binding fragment thereof. In some aspects, the one or more tertiary doses comprise: (a) 350 mg anti-PD-1 antibody or antigen-binding fragment thereof and (b) 400 mg, 800 mg, 1000 mg, 1400 mg, 1600 mg or 2000 mg anti-LAG-3 antibody or antigen-binding fragment thereof.

The various aspects provided herein can be applied to any one or each of the other methods provided herein. Illustratively, in any one of the above-described methods, one dose of the anti-PD-1 antibody or antigen-binding fragment thereof comprises 50 to 1500 mg, or 350 mg. In any one of the above-described methods, one dose of the anti-LAG-3 antibody or antigen-binding fragment thereof comprises between 50 to 8000 mg, or 400 mg, or 1600 mg. In any one of the above-described methods, the anti-LAG-3 antibody or antigen-binding fragment thereof is administered prior to, concurrent with or after the anti-PD-1 antibody or antigen-binding fragment thereof. In any one of the above-described methods, the anti-LAG-3 antibody or antigen-binding fragment thereof is administered prior to the anti-PD-1 antibody or antigen-binding fragment thereof. In any one of the above-described methods, the anti-LAG-3 antibody or antigen-binding fragment thereof is administered the same day as the anti-PD-1 antibody or antigen-binding fragment thereof. In any one of the above-described methods, two or more doses of the anti-LAG-3 antibody or antigen-binding fragment thereof are administered in combination with two or more doses of the anti-PD-1 antibody or antigen-binding fragment thereof. In some embodiments, each dose of the anti-PD-1 antibody or antigen-binding fragment thereof comprises 350 mg. In some embodiments, each dose of the anti-LAG-3 antibody or antigen-binding fragment thereof comprises between 50 and 8000 mg. In some embodiments, each dose of the anti-LAG-3 antibody or antigen-binding fragment thereof comprises 1600 mg. In some embodiments, each dose of the anti-LAG-3 antibody or antigen-binding fragment thereof comprises 400 mg. In any one of the above-described methods, each dose of the anti-PD-1 antibody or antigen-binding fragment thereof comprises 200 mg, 250 mg or 350 mg and each dose of the anti-LAG-3 antibody or antigen-binding fragment thereof comprises 400 mg, 800 mg, 1000 mg, 1400 mg, or 1600 mg. In some aspects, each dose of the anti-PD-1 antibody or antigen-binding fragment thereof is administered 0.5 weeks to 12 weeks after the immediately preceding dose. In some aspects, each dose of the anti-LAG-3 antibody or antigen-binding fragment thereof is administered 0.5 weeks to 12 weeks after the immediately preceding dose. 18. In some aspects, each dose of the anti-PD-1 antibody or antigen-binding fragment thereof is administered once in six weeks. In some aspects, n each dose of the anti-LAG-3 antibody or antigen-binding fragment thereof is administered once in six weeks. In some aspects, each dose of the anti-PD-1 antibody or antigen-binding fragment thereof is administered once in three weeks. In some aspects, each dose of the anti-LAG-3 antibody or antigen-binding fragment thereof is administered once in three weeks. In any one of the above-described methods, antibodies are administered intravenously, subcutaneously, or intraperitoneally. In some aspects, the melanoma is unresectable locally advanced melanoma. In some aspects, the melanoma is unresectable metastatic melanoma. In some aspects, the melanoma is completely resected high-risk melanoma.

In some embodiments, the patient is further selected as having one or more of the following criteria:
(i) at least 12 years of age on the date of providing informed consent;
(ii) either stage IIC, III, or stage IV per AJCC 8th edition (Amin, 2017) and have histologically confirmed melanoma that is completely surgically resected in order to be eligible;
(iii) patients with stage IIIA disease must have at least 1 lymph node micrometastasis measuring >1 mm in greatest diameter;
(iv) Stage IIC melanoma confirmed by a pathologically negative Sentinel lymph node biopsy (SLNB) specimen and no evidence of regional or distant metastasis;
(v) complete surgical resection performed within 12 weeks prior to randomization, and treatment may occur only after satisfactory wound healing from the surgery;
(vi) disease-free status as documented by a complete physical examination and imaging studies prior to treatment;
(vii) patient shows ≥1% LAG3 in the tumor tissue as determined by IHC or iPET; and
(viii) patients must not have received systemic anti-cancer therapy or radiation therapy for melanoma in the previous 5 years.

In some embodiments, the patient is further selected as having one or more of the following criteria:
(1) ≥12 years of age;
(2) Stage IIc, III or IV (all M-stages) and histologically confirmed melanoma, completely resected <12 weeks prior to randomization;
(3) no prior systemic anti-cancer therapy or radiation therapy for melanoma in the previous 5 years;
(4) shows ≥1% LAG3 in the tumor tissue as determined by IHC or iPET;
(5) no evidence of metastatic disease; and
(6) an Eastern Cooperative Oncology Group performance status (PS) of 0 or 1 (for adult pts), Karnofsky PS>70 (pts>16 years) or Lansky PS>70 (pts<16 years).

According to any one of the methods provided herein, the treatment produces a therapeutic effect selected from the group consisting of delay in melanoma growth, reduction in melanoma cell number, melanoma regression, increase in survival, partial response, and complete response. In some aspects, melanoma growth is delayed by at least 10 days as compared to an untreated subject. In some aspects, melanoma growth is inhibited by at least 50% as compared to an untreated subject. In some aspects, melanoma growth is inhibited by at least 20% as compared to a subject administered with either antibody as monotherapy.

In some embodiments, methods provided herein can further comprise administering to the subject an additional therapeutic agent or therapy, wherein the additional therapeutic agent or therapy is selected from the group consisting of radiation, surgery, a chemotherapeutic agent, a cancer vaccine, a PD-L1 inhibitor, a CTLA-4 inhibitor, a TIM3 inhibitor, a BTLA inhibitor, a TIGIT inhibitor, a CD47 inhibitor, a CD28 agonist, a CD38 inhibitor, an indoleamine-2,3-dioxygenase (IDO) inhibitor, a vascular endothelial growth factor (VEGF) antagonist, an angiopoietin-2 (Ang2) inhibitor, a transforming growth factor beta (TGFβ) inhibitor, an epidermal growth factor receptor (EGFR) inhibitor, an antibody to a tumor-specific antigen, Bacillus Calmette-Guerin vaccine, granulocyte-macrophage colony-stimulating factor, an oncolytic virus, a cytotoxin, an interleukin 6 receptor (IL-6R) inhibitor, an interleukin 4 receptor (IL-4R) inhibitor, an IL-10 inhibitor, IL-2, IL-7, IL-21, IL-12, IL-15, an antibody-drug conjugate, a GITR agonist, a 4-1BB agonist, CD20xCD3 bispecific antibody, MUC16xCD3 bispecific antibody, and an anti-inflammatory drug.

In some embodiments, the additional therapeutic agent is an anti-PD-1 antibody or antigen-binding fragment thereof comprises the heavy chain complementarity determining regions (HCDR1, HCDR2 and HCDR3) of a heavy chain variable region (HCVR) and three light chain complementarity determining regions (LCDR1, LCDR2 and LCDR3) of a light chain variable region (LCVR), wherein HCDR1 comprises the amino acid sequence of SEQ ID NO: 3; HCDR2 comprises the amino acid sequence of SEQ ID NO: 4; HCDR3 comprises the amino acid sequence of SEQ ID NO: 5; LCDR1 comprises the amino acid sequence of SEQ ID NO: 6; LCDR2 comprises the amino acid sequence of SEQ ID NO: 7; and LCDR3 comprises the amino acid sequence of SEQ ID NO: 8. In some aspects, the HCVR comprises the amino acid sequence of SEQ ID NO: 1 and the LCVR comprises the amino acid sequence of SEQ ID NO: 2. In some aspects, the anti-PD-1 antibody comprises a heavy chain comprising the amino acid sequence of SEQ ID NO: 9 and a light chain comprising the amino acid sequence of SEQ ID NO: 10.

In some embodiments, the anti-LAG-3 antibody or antigen-binding fragment thereof comprises the heavy chain CDRs (HCDR1, HCDR2 and HCDR3) of a HCVR and three light chain CDRs (LCDR1, LCDR2 and LCDR3) of a LCVR, wherein HCDR1 comprises the amino acid sequence of SEQ ID NO: 13; HCDR2 comprises the amino acid sequence of SEQ ID NO: 14; HCDR3 comprises the amino acid sequence of SEQ ID NO: 15; LCDR1 comprises the amino acid sequence of SEQ ID NO: 16; LCDR2 comprises the amino acid sequence of SEQ ID NO: 17; and LCDR3 comprises the amino acid sequence of SEQ ID NO: 18. In some aspects, the HCVR comprises the amino acid sequence of SEQ ID NO: 11 and the LCVR comprises the amino acid sequence of SEQ ID NO: 12. In some aspects, the anti-LAG-3 antibody comprises a heavy chain comprising the amino acid sequence of SEQ ID NO: 19 and a light chain comprising the amino acid sequence of SEQ ID NO: 20.

According to any one of the methods provided herein, the inhibition is more efficacious than administration of either antibody as a monotherapy.

Other embodiments will become apparent from a review of the ensuing detailed description.

### BRIEF DESCRIPTION OF THE FIGURES

FIG. 1A and FIG. 1B depict a study flow diagram for Example 1.

FIG. 2 depicts a study flow diagram for Example 2.

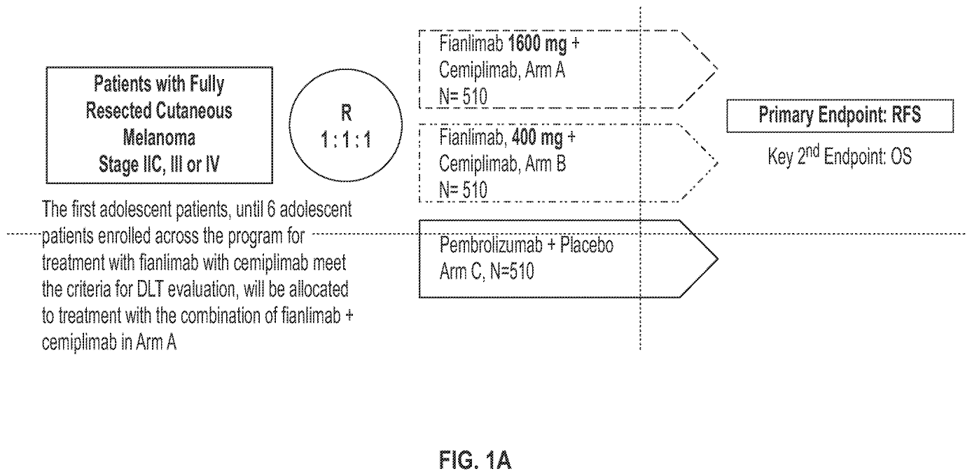

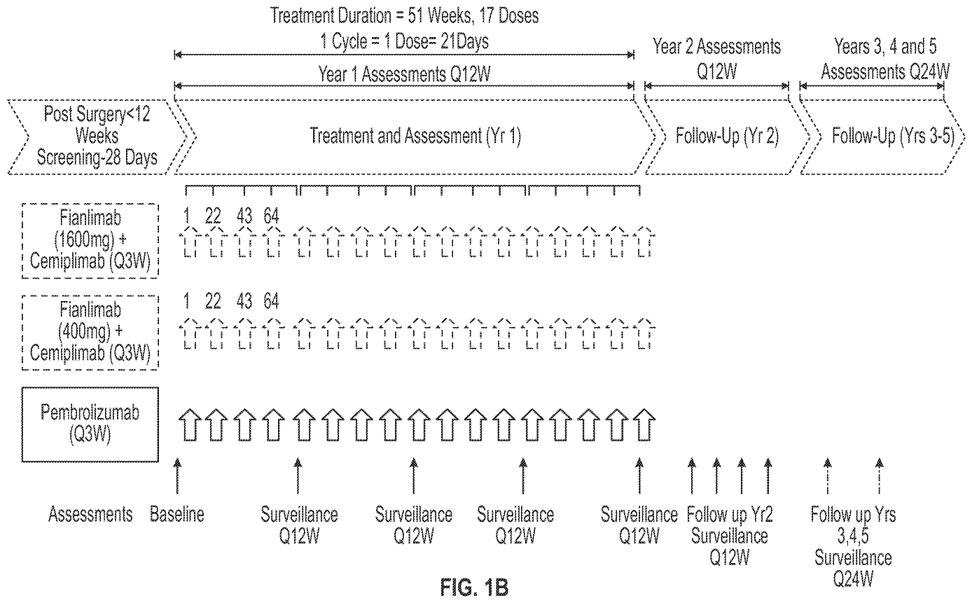

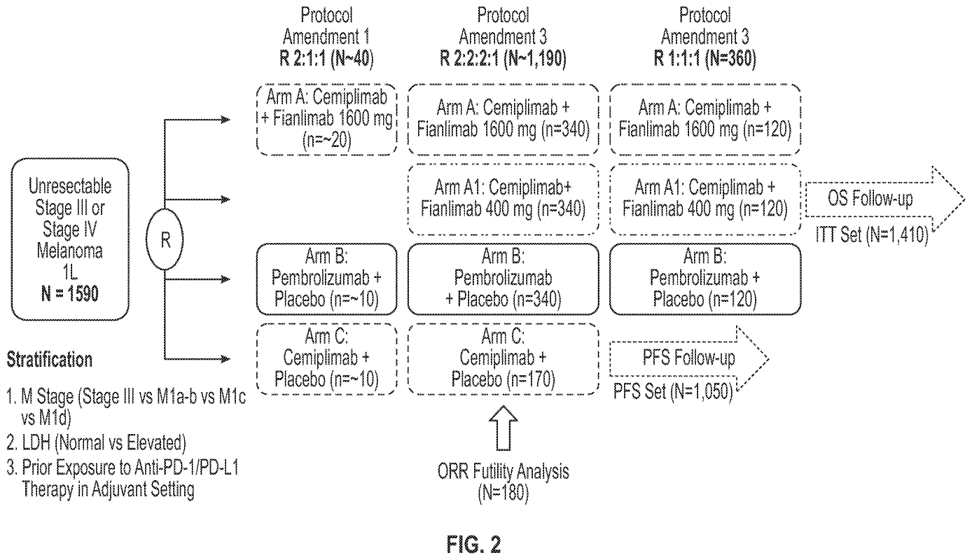

### DETAILED DESCRIPTION

It is to be understood that this invention is not limited to particular methods and experimental conditions described, as such methods and conditions may vary. It is also to be understood that the terminology used herein is for the purpose of describing particular embodiments only, and is not intended to be limiting, since the scope of the present disclosure will be limited only by the appended claims.

Unless defined otherwise, all technical and scientific terms used herein have the same meaning as commonly understood by one of ordinary skill in the art to which this disclosure belongs. As used herein, the term “about,” when used in reference to a particular recited numerical value, means that the value may vary from the recited value by no more than 1%. For example, as used herein, the expression “about 100” includes 99 and 101 and all values in between (e.g., 99.1, 99.2, 99.3, 99.4, etc.).

The term “antibody,” as used herein, includes immunoglobulin molecules comprising four polypeptide chains, two heavy (H) chains and two light (L) chains inter-connected by disulfide bonds, as well as multimers thereof (e.g., IgM). In a typical antibody, each heavy chain comprises a heavy chain variable region (abbreviated herein as HCVR or V_{H}) and a heavy chain constant region. The heavy chain constant region comprises three domains, C_{H1}, C_{H2} and C_{H3}. Each light chain comprises a light chain variable region (abbreviated herein as LCVR or V_{L}) and a light chain constant region. The light chain constant region comprises one domain (C_{L1}). The V_{H} and V_{L} regions can be further subdivided into regions of hypervariability, termed complementarity determining regions (CDRs), interspersed with regions that are more conserved, termed framework regions (FR). Each V_{H} and V_{L} is composed of three CDRs and four FRs, arranged from amino-terminus to carboxy-terminus in the following order: FR1, CDR1, FR2, CDR2, FR3, CDR3, FR4. In different embodiments of the disclosure, the FRs of the anti-IL-4R antibody (or antigen-binding portion thereof) may be identical to the human germline sequences, or may be naturally or artificially modified. An amino acid consensus sequence may be defined based on a side-by-side analysis of two or more CDRs.

The term “antibody,” as used herein, also includes antigen-binding fragments of full antibody molecules. The terms “antigen-binding portion” of an antibody, “antigen-binding fragment” of an antibody, and the like, as used herein, include any naturally occurring, enzymatically obtainable, synthetic, or genetically engineered polypeptide or glycoprotein that specifically binds an antigen to form a complex. Antigen-binding fragments of an antibody may be derived, e.g., from full antibody molecules using any suitable standard techniques such as proteolytic digestion or recombinant genetic engineering techniques involving the manipulation and expression of DNA encoding antibody variable and optionally constant domains. Such DNA is known and/or is readily available from, e.g., commercial sources, DNA libraries (including, e.g., phage-antibody libraries), or can be synthesized. The DNA may be sequenced and manipulated chemically or by using molecular biology techniques, for example, to arrange one or more variable and/or constant domains into a suitable configuration, or to introduce codons, create cysteine residues, modify, add or delete amino acids, etc.

Non-limiting examples of antigen-binding fragments include: (i) Fab fragments; (ii) F(ab′)2 fragments; (iii) Fd fragments; (iv) Fv fragments; (v) single-chain Fv (scFv) molecules; (vi) dAb fragments; and (vii) minimal recognition units consisting of the amino acid residues that mimic the hypervariable region of an antibody (e.g., an isolated complementarity determining region (CDR) such as a CDR3 peptide), or a constrained FR3-CDR3-FR4 peptide. Other engineered molecules, such as domain-specific antibodies, single domain antibodies, domain-deleted antibodies, chimeric antibodies, CDR-grafted antibodies, diabodies, triabodies, tetrabodies, minibodies, nanobodies (e.g. monovalent nanobodies, bivalent nanobodies, etc.), small modular immunopharmaceuticals (SMIPs), and shark variable IgNAR domains, are also encompassed within the expression “antigen-binding fragment,” as used herein.

An antigen-binding fragment of an antibody will typically comprise at least one variable domain. The variable domain may be of any size or amino acid composition and will generally comprise at least one CDR which is adjacent to or in frame with one or more framework sequences. In antigen-binding fragments having a V_{H} domain associated with a V_{L} domain, the V_{H} and V_{L} domains may be situated relative to one another in any suitable arrangement. For example, the variable region may be dimeric and contain V_{H}-V_{H}, V_{H}-V_{L} or V_{L}-V_{L} dimers. Alternatively, the antigen-binding fragment of an antibody may contain a monomeric V_{H} or V_{L} domain.

In certain embodiments, an antigen-binding fragment of an antibody may contain at least one variable domain covalently linked to at least one constant domain. Non-limiting, exemplary configurations of variable and constant domains that may be found within an antigen-binding fragment of an antibody of the present disclosure include: (i) V_{H}-C_{H1}; (ii) V_{H}-C_{H2}; (iii) V_{H}-C_{H3}; (iv) V_{H}-C_{H1}-C_{H2}; (v) V_{H}-C_{H1}-C_{H2}-C_{H3}; (vi) V_{H}-C_{H2}-C_{H3}; (vii) V_{H}-C_{L}; (viii) V_{L}-C_{H1}; (ix) V_{L}-C_{H2}; (x) V_{L}-C_{H3}; (xi) V_{L}-C_{H1}-C_{H2}; (xii) V_{L}-C_{H1}-C_{H2}-C_{H3}; (xiii) V_{L}-C_{H2}-CH₃; and (xiv) V_{L}-C_{L}. In any configuration of variable and constant domains, including any of the exemplary configurations listed above, the variable and constant domains may be either directly linked to one another or may be linked by a full or partial hinge or linker region. A hinge region may consist of at least 2 (e.g., 5, 10, 15, 20, 40, 60 or more) amino acids which result in a flexible or semi-flexible linkage between adjacent variable and/or constant domains in a single polypeptide molecule. Moreover, an antigen-binding fragment of an antibody of the present disclosure may comprise a homo-dimer or hetero-dimer (or other multimer) of any of the variable and constant domain configurations listed above in non-covalent association with one another and/or with one or more monomeric V_{H} or V_{L} domain (e.g., by disulfide bond(s)).

The term “antibody,” as used herein, also includes multispecific (e.g., bispecific) antibodies. A multispecific antibody or antigen-binding fragment of an antibody will typically comprise at least two different variable domains, wherein each variable domain is capable of specifically binding to a separate antigen or to a different epitope on the same antigen. Any multispecific antibody format may be adapted for use in the context of an antibody or antigen-binding fragment of an antibody of the present disclosure using routine techniques available in the art. For example, the present disclosure includes methods comprising the use of bispecific antibodies wherein one arm of an immunoglobulin is specific for PD-1 or LAG-3, or fragments thereof, and the other arm of the immunoglobulin is specific for a second therapeutic target or is conjugated to a therapeutic moiety. Exemplary bispecific formats that can be used in the context of the present disclosure include, without limitation, e.g., scFv-based or diabody bispecific formats, IgG-scFv fusions, dual variable domain (DVD)-Ig, Quadroma, knobs-into-holes, common light chain (e.g., common light chain with knobs-into-holes, etc.), CrossMab, CrossFab, (SEED) body, leucine zipper, Duobody, IgG1/IgG2, dual acting Fab (DAF)-IgG, and Mab² bispecific formats (see, e.g., Klein et al. 2012, mAbs 4:6, 1-11, and references cited therein, for a review of the foregoing formats). Bispecific antibodies can also be constructed using peptide/nucleic acid conjugation, e.g., wherein unnatural amino acids with orthogonal chemical reactivity are used to generate site-specific antibody-oligonucleotide conjugates which then self-assemble into multimeric complexes with defined composition, valency and geometry. (See, e.g., Kazane et al., J. Am. Chem. Soc. [Epub: Dec. 4, 2012]).

The antibodies used in the methods of the present disclosure may be human antibodies. The term “human antibody,” as used herein, is intended to include antibodies having variable and constant regions derived from human germline immunoglobulin sequences. The human antibodies of the disclosure may nonetheless include amino acid residues not encoded by human germline immunoglobulin sequences (e.g., mutations introduced by random or site-specific mutagenesis in vitro or by somatic mutation in vivo), for example in the CDRs and in particular CDR3. However, the term “human antibody,” as used herein, is not intended to include antibodies in which CDR sequences derived from the germline of another mammalian species, such as a mouse, have been grafted onto human framework sequences.

The antibodies used in the methods of the present disclosure may be recombinant human antibodies. The term “recombinant human antibody,” as used herein, is intended to include all human antibodies that are prepared, expressed, created or isolated by recombinant means, such as antibodies expressed using a recombinant expression vector transfected into a host cell (described further below), antibodies isolated from a recombinant, combinatorial human antibody library (described further below), antibodies isolated from an animal (e.g., a mouse) that is transgenic for human immunoglobulin genes (see e.g., Taylor et al. (1992) Nucl. Acids Res. 20:6287-6295), or antibodies prepared, expressed, created or isolated by any other means that involves splicing of human immunoglobulin gene sequences to other DNA sequences. Such recombinant human antibodies have variable and constant regions derived from human germline immunoglobulin sequences. In certain embodiments, however, such recombinant human antibodies are subjected to in vitro mutagenesis (or, when an animal transgenic for human Ig sequences is used, in vivo somatic mutagenesis) and thus the amino acid sequences of the V_{H} and V_{L} regions of the recombinant antibodies are sequences that, while derived from and related to human germline V_{H} and V_{L} sequences, may not naturally exist within the human antibody germline repertoire in vivo.

Although any methods and materials similar or equivalent to those described herein can be used in the practice of the present disclosure, exemplary methods and materials are now described. All publications mentioned herein are incorporated herein by reference in their entirety.

### General Methods

Standard methods in molecular biology are described Sambrook, Fritsch and Maniatis (1982 & 1989 2^{nd} Edition, 2001 3^{rd} Edition) Molecular Cloning, A Laboratory Manual, Cold Spring Harbor Laboratory Press, Cold Spring Harbor, N.Y.; Sambrook and Russell (2001) Molecular Cloning, 3rd ed., Cold Spring Harbor Laboratory Press, Cold Spring Harbor, N.Y.; Wu (1993) Recombinant DNA, Vol. 217, Academic Press, San Diego, Calif.). Standard methods also appear in Ausbel, et al. (2001) Current Protocols in Molecular Biology, Vols. 1-4, John Wiley and Sons, Inc. New York, N.Y., which describes cloning in bacterial cells and DNA mutagenesis (Vol. 1), cloning in mammalian cells and yeast (Vol. 2), glycoconjugates and protein expression (Vol. 3), and bioinformatics (Vol. 4).

Methods for protein purification including immunoprecipitation, chromatography, electrophoresis, centrifugation, and crystallization are described (Coligan, et al. (2000) Current Protocols in Protein Science, Vol. 1, John Wiley and Sons, Inc., New York). Chemical analysis, chemical modification, post-translational modification, production of fusion proteins, glycosylation of proteins are described (see, e.g., Coligan, et al. (2000) Current Protocols in Protein Science, Vol. 2, John Wiley and Sons, Inc., New York; Ausubel, et al. (2001) Current Protocols in Molecular Biology, Vol. 3, John Wiley and Sons, Inc., NY, NY, pp. 16.0.5-16.22.17; Sigma-Aldrich, Co. (2001) Products for Life Science Research, St. Louis, Mo.; pp. 45-89; Amersham Pharmacia Biotech (2001) BioDirectory, Piscataway, N.J., pp. 384-391). Production, purification, and fragmentation of polyclonal and monoclonal antibodies are described (Coligan, et al. (2001) Current Protocols in Immunology, Vol. 1, John Wiley and Sons, Inc., New York; Harlow and Lane (1999) Using Antibodies, Cold Spring Harbor Laboratory Press, Cold Spring Harbor, N.Y.; Harlow and Lane, supra). Standard techniques for characterizing ligand/receptor interactions are available (see, e.g., Coligan, et al. (2001) Current Protocols in Immunology, Vol. 4, John Wiley, Inc., New York).

Monoclonal, polyclonal, and humanized antibodies can be prepared (see, e.g., Sheperd and Dean (eds.) (2000) Monoclonal Antibodies, Oxford Univ. Press, New York, N.Y.; Kontermann and Dubel (eds.) (2001) Antibody Engineering, Springer-Verlag, New York; Harlow and Lane (1988) Antibodies A Laboratory Manual, Cold Spring Harbor Laboratory Press, Cold Spring Harbor, N.Y., pp. 139-243; Carpenter, et al. (2000) J. Immunol. 165:6205; He, et al. (1998) J. Immunol. 160:1029; Tang et al. (1999) J. Biol. Chem. 274:27371-27378; Baca et al. (1997) J. Biol. Chem. 272:10678-10684; Chothia et al. (1989) Nature 342:877-883; Foote and Winter (1992) J. Mol. Biol. 224:487-499; U.S. Pat. No. 6,329,511).

An alternative to humanization is to use human antibody libraries displayed on phage or human antibody libraries in transgenic mice (Vaughan et al. (1996) Nature Biotechnol. 14:309-314; Barbas (1995) Nature Medicine 1:837-839; Mendez et al. (1997) Nature Genetics 15:146-156; Hoogenboom and Chames (2000) Immunol. Today 21:371-377; Barbas et al. (2001) Phage Display: A Laboratory Manual, Cold Spring Harbor Laboratory Press, Cold Spring Harbor, N.Y.; Kay et al. (1996) Phage Display of Peptides and Proteins: A Laboratory Manual, Academic Press, San Diego, Calif.; de Bruin et al. (1999) Nature Biotechnol. 17:397-399). Single chain antibodies and diabodies are described (see, e.g., Malecki et al. (2002) Proc. Natl. Acad. Sci. USA 99:213-218; Conrath et al. (2001) J. Biol. Chem. 276:7346-7350; Desmyter et al. (2001) J. Biol. Chem. 276:26285-26290; Hudson and Kortt (1999) J. Immunol. Methods 231:177-189; and U.S. Pat. No. 4,946,778). Bifunctional antibodies are provided (see, e.g., Mack, et al. (1995) Proc. Natl. Acad. Sci. USA 92:7021-7025; Carter (2001) J. Immunol. Methods 248:7-15; Volkel, et al. (2001) Protein Engineering 14:815-823; Segal, et al. (2001) J. Immunol. Methods 248:1-6; Brennan, et al. (1985) Science 229:81-83; Raso, et al. (1997) J. Biol. Chem. 272:27623; Morrison (1985) Science 229:1202-1207; Traunecker, et al. (1991) EMBO J. 10:3655-3659; and U.S. Pat. Nos. 5,932,448, 5,532,210, and 6,129,914). Fully human antibodies may also be developed in genetically engineered mice such as the VelociMouse. See e.g., DeChiara et al., Producing fully ES cell-derived mice from eight-cell stage embryo injections, Methods Enzymol, 476:285-94 (2010); Dechiara et al., VelociMouse: fully ES cell-derived FO-generation mice obtained from the injection of ES cells into eight-cell-stage embryos. Methods Mol Biol, 530:311-24 (2009); U.S. Pat. Nos. 7,576,259; 7,659,442; or 7294754, and US2008/0078000A1.

Purification of antigen is not typically necessary for the generation of antibodies. Animals can be immunized with cells bearing the antigen of interest. Splenocytes can then be isolated from the immunized animals, and the splenocytes can fused with a myeloma cell line to produce a hybridoma (see, e.g., Meyaard et al. (1997) Immunity 7:283-290; Wright et al. (2000) Immunity 13:233-242; Preston et al., supra; Kaithamana et al. (1999) J. Immunol. 163:5157-5164).

Antibodies can be conjugated, e.g., to small drug molecules, enzymes, liposomes, polyethylene glycol (PEG). Antibodies are useful for therapeutic, diagnostic, kit or other purposes, and include antibodies coupled, e.g., to dyes, radioisotopes, enzymes, or metals, e.g., colloidal gold (see, e.g., Le Doussal et al. (1991) J. Immunol. 146:169-175; Gibellini et al. (1998) J. Immunol. 160:3891-3898; Hsing and Bishop (1999) J. Immunol. 162:2804-2811; Everts et al. (2002) J. Immunol. 168:883-889).

Methods for flow cytometry, including fluorescence activated cell sorting (FACS), are available (see, e.g., Owens, et al. (1994) Flow Cytometry Principles for Clinical Laboratory Practice, John Wiley and Sons, Hoboken, N.J.; Givan (2001) Flow Cytometry, 2nd ed.; Wiley-Liss, Hoboken, N.J.; Shapiro (2003) Practical Flow Cytometry, John Wiley and Sons, Hoboken, N.J.). Fluorescent reagents suitable for modifying nucleic acids, including nucleic acid primers and probes, polypeptides, and antibodies, for use, e.g., as diagnostic reagents, are available (Molecular Probes (2003) Catalogue, Molecular Probes, Inc., Eugene, Oreg.; Sigma-Aldrich (2003) Catalogue, St. Louis, Mo.).

Standard methods of histology of the immune system are described (see, e.g., Muller-Harmelink (ed.) (1986) Human Thymus: Histopathology and Pathology, Springer Verlag, New York, N.Y.; Hiatt, et al. (2000) Color Atlas of Histology, Lippincott, Williams, and Wilkins, Phila, Pa.; Louis, et al. (2002) Basic Histology: Text and Atlas, McGraw-Hill, New York, N.Y.).

Software packages and databases for determining, e.g., antigenic fragments, leader sequences, protein folding, functional domains, glycosylation sites, and sequence alignments, are available (see, e.g., GenBank, Vector NTI® Suite (Informax, Inc, Bethesda, Md.); GCG Wisconsin Package (Accelrys, Inc., San Diego, Calif.); DeCypher® (TimeLogic Corp., Crystal Bay, Nev.); Menne, et al. (2000) Bioinformatics 16: 741-742; Menne, et al. (2000) Bioinformatics Applications Note 16:741-742; Wren, et al. (2002) Comput. Methods Programs Biomed. 68:177-181; von Heijne (1983) Eur. J. Biochem. 133:17-21; von Heijne (1986) Nucleic Acids Res. 14:4683-4690).

Methods and techniques for identifying CDRs within HCVR and LCVR amino acid sequences are well known in the art and can be used to identify CDRs within the specified HCVR and/or LCVR amino acid sequences disclosed herein. Exemplary conventions that can be used to identify the boundaries of CDRs include, e.g., the Kabat definition, the Chothia definition, and the AbM definition. In general terms, the Kabat definition is based on sequence variability, the Chothia definition is based on the location of the structural loop regions, and the AbM definition is a compromise between the Kabat and Chothia approaches. See, e.g., Kabat, “Sequences of Proteins of Immunological Interest,” National Institutes of Health, Bethesda, Md. (1991); Al-Lazikani et al., J. Mol. Biol. 273:927-948 (1997); and Martin et al., Proc. Natl. Acad. Sci. USA 86:9268-9272 (1989). Public databases are also available for identifying CDR sequences within an antibody.

### PD-1 Inhibitors

According to certain exemplary embodiments of the present disclosure, the methods comprise administering a therapeutically effective amount of an anti-PD-1 antibody or antigen-binding fragment thereof. The term “PD-1” refers to the programmed death-1 protein, a T-cell co-inhibitor, also known as CD279. The amino acid sequence of full-length PD-1 is provided in GenBank as accession number NP_005009.2. PD-1 is a member of the CD28/CTLA-4/ICOS family of T-cell co-inhibitors. PD-1 is a 288-amino acid protein with an extracellular N-terminal domain which is IgV-like, a transmembrane domain and an intracellular domain containing an immunoreceptor tyrosine-based inhibitory (ITIM) motif and an immunoreceptor tyrosine-based switch (ITSM) motif (Chattopadhyay et al 2009, Immunol. Rev.). The PD-1 receptor has two ligands, PD-ligand-1 (PD-L1) and PD-L2.

PD-L1 is a 290 amino acid protein with an extracellular IgV-like domain, a transmembrane domain and a highly conserved intracellular domain of approximately 30 amino acids. PD-L1 is constitutively expressed on many cells such as antigen presenting cells (e.g., dendritic cells, macrophages, and B-cells) and on hematopoietic and non-hematopoietic cells (e.g., vascular endothelial cells, pancreatic islets, and sites of immune privilege). PD-L1 is also expressed on a wide variety of tumors, virally-infected cells and autoimmune tissue, and is a component of the immunosuppressive milieu (Ribas 2012, NEJM 366: 2517-2519).

PD-1 inhibitors include antibodies and antigen-binding fragments thereof and other substances (e.g., peptides and small molecules) that specifically bind to PD-1 and antagonize one or more biological activities of PD-1. Molecules that specifically bind to PD-1 may be referred to as “anti-PD-1”. In an embodiment of the disclosure, the PD-1 inhibitor is an antibody or antigen-binding fragment thereof that binds PD-L1 or PD-L2.

In an embodiment of the disclosure, the PD-1 inhibitor is an antibody or antigen-binding fragment thereof as set forth in U.S. Pat. No. 9,987,500.

According to certain embodiments, the antibodies used in the methods of the present disclosure specifically bind PD-1. The term “specifically binds,” or the like, means that an antibody or antigen-binding fragment thereof forms a complex with an antigen that is relatively stable under physiologic conditions. Methods for determining whether an antibody specifically binds to an antigen are well known in the art and include, for example, equilibrium dialysis, surface plasmon resonance, and the like. For example, an antibody that “specifically binds” PD-1, as used in the context of the present disclosure, includes antibodies that bind PD-1 or portion thereof with a K_{D} of less than about 500 nM, less than about 300 nM, less than about 200 nM, less than about 100 nM, less than about 90 nM, less than about 80 nM, less than about 70 nM, less than about 60 nM, less than about 50 nM, less than about 40 nM, less than about 30 nM, less than about 20 nM, less than about 10 nM, less than about 5 nM, less than about 4 nM, less than about 3 nM, less than about 2 nM, less than about 1 nM or less than about 0.5 nM, as measured in a surface plasmon resonance assay. An isolated antibody that specifically binds human PD-1 may, however, have cross-reactivity to other antigens, such as PD-1 molecules from other (non-human) species.

According to certain exemplary embodiments of the present disclosure, the anti-PD-1 antibody, or antigen-binding fragment thereof comprises a heavy chain variable region (HCVR), light chain variable region (LCVR), and/or complementarity determining regions (CDRs) comprising any of the amino acid sequences of the anti-PD-1 antibodies as set forth in U.S. Pat. No. 9,987,500.

In certain exemplary embodiments, the anti-PD-1 antibody or antigen-binding fragment thereof that can be used in the context of the methods of the present disclosure comprises the heavy chain complementarity determining regions (HCDRs) of a heavy chain variable region (HCVR) comprising the amino acid sequence of SEQ ID NO: 1 and the light chain complementarity determining regions (LCDRs) of a light chain variable region (LCVR) comprising the amino acid sequence of SEQ ID NO: 2. According to certain embodiments, the anti-PD-1 antibody or antigen-binding fragment thereof comprises three HCDRs (HCDR1, HCDR2 and HCDR3) and three LCDRs (LCDR1, LCDR2 and LCDR3), wherein the HCDR1 comprises the amino acid sequence of SEQ ID NO: 3; the HCDR2 comprises the amino acid sequence of SEQ ID NO: 4; the HCDR3 comprises the amino acid sequence of SEQ ID NO: 5; the LCDR1 comprises the amino acid sequence of SEQ ID NO: 6; the LCDR2 comprises the amino acid sequence of SEQ ID NO: 7; and the LCDR3 comprises the amino acid sequence of SEQ ID NO: 8. In yet other embodiments, the anti-PD-1 antibody or antigen-binding fragment thereof comprises an HCVR comprising SEQ ID NO: 1 and an LCVR comprising SEQ ID NO: 2. In certain embodiments, the methods of the present disclosure comprise the use of an anti-PD-1 antibody, wherein the antibody comprises a heavy chain comprising the amino acid sequence of SEQ ID NO: 9. In some embodiments, the anti-PD-1 antibody comprises a light chain comprising the amino acid sequence of SEQ ID NO: 10. An exemplary antibody comprising a heavy chain variable region comprising the amino acid sequence of SEQ ID NO: 1 and a light chain variable region comprising the amino acid sequence of SEQ ID NO: 2 is the fully human anti-PD-1 antibody known as REGN2810 (cemiplimab; LIBTAYO®).

According to certain exemplary embodiments, the methods of the present disclosure comprise the use of REGN2810, or a bioequivalent thereof. The term “bioequivalent”, as used herein, refers to anti-PD-1 antibodies or PD-1-binding proteins or fragments thereof that are pharmaceutical equivalents or pharmaceutical alternatives whose rate and/or extent of absorption do not show a significant difference with that of REGN2810 when administered at the same molar dose under similar experimental conditions, either single dose or multiple dose. In the context of the disclosure, the term refers to antigen-binding proteins that bind to PD-1 which do not have clinically meaningful differences with REGN2810 in their safety, purity and/or potency.

Other anti-PD-1 antibodies that can be used in the context of the methods of the present disclosure include, e.g., the antibodies referred to and known in the art as nivolumab (U.S. Pat. No. 8,008,449), pembrolizumab (U.S. Pat. No. 8,354,509), MED10608 (U.S. Pat. No. 8,609,089), pidilizumab (U.S. Pat. No. 8,686,119), or any of the anti-PD-1 antibodies as set forth in U.S. Pat. Nos. 6,808,710, 7,488,802, 8,168,757, 8,354,509, 8,779,105, or 8900587. In an embodiment of the disclosure, a PD-1 inhibitor is as set forth in any of U.S. 20110008369, U.S. 20130017199, U.S. 20130022595, WO2006121168, WO20091154335, WO2012145493, WO2013014668, WO2009101611, EP2262837, and EP2504028.

The anti-PD-1 antibodies used in the context of the methods of the present disclosure may have pH-dependent binding characteristics. For example, an anti-PD-1 antibody for use in the methods of the present disclosure may exhibit reduced binding to PD-1 at acidic pH as compared to neutral pH. Alternatively, an anti-PD-1 antibody of the disclosure may exhibit enhanced binding to its antigen at acidic pH as compared to neutral pH. The expression “acidic pH” includes pH values less than about 6.2, e.g., about 6.0, 5.95, 5.9, 5.85, 5.8, 5.75, 5.7, 5.65, 5.6, 5.55, 5.5, 5.45, 5.4, 5.35, 5.3, 5.25, 5.2, 5.15, 5.1, 5.05, 5.0, or less. As used herein, the expression “neutral pH” means a pH of about 7.0 to about 7.4. The expression “neutral pH” includes pH values of about 7.0, 7.05, 7.1, 7.15, 7.2, 7.25, 7.3, 7.35, and 7.4.

In certain instances, “reduced binding to PD-1 at acidic pH as compared to neutral pH” is expressed in terms of a ratio of the K_{D} value of the antibody binding to PD-1 at acidic pH to the K_{D} value of the antibody binding to PD-1 at neutral pH (or vice versa). For example, an antibody or antigen-binding fragment thereof may be regarded as exhibiting “reduced binding to PD-1 at acidic pH as compared to neutral pH” for purposes of the present disclosure if the antibody or antigen-binding fragment thereof exhibits an acidic/neutral K_{D} ratio of about 3.0 or greater. In certain exemplary embodiments, the acidic/neutral K_{D} ratio for an antibody or antigen-binding fragment of the present disclosure can be about 3.0, 3.5, 4.0, 4.5, 5.0, 5.5, 6.0, 6.5, 7.0, 7.5, 8.0, 8.5, 9.0, 9.5, 10.0, 10.5, 11.0, 11.5, 12.0, 12.5, 13.0, 13.5, 14.0, 14.5, 15.0, 20.0, 25.0, 30.0, 40.0, 50.0, 60.0, 70.0, 100.0, or greater.

Antibodies with pH-dependent binding characteristics may be obtained, e.g., by screening a population of antibodies for reduced (or enhanced) binding to a particular antigen at acidic pH as compared to neutral pH. Additionally, modifications of the antigen-binding domain at the amino acid level may yield antibodies with pH-dependent characteristics. For example, by substituting one or more amino acids of an antigen-binding domain (e.g., within a CDR) with a histidine residue, an antibody with reduced antigen-binding at acidic pH relative to neutral pH may be obtained. As used herein, the expression “acidic pH” means a pH of 6.0 or less.

### LAG-3 Inhibitors

The term “LAG-3” refers to the lymphocyte activation gene-3 protein, an immune checkpoint receptor or T cell co-inhibitor, also known as CD223. The amino acid sequence of full-length LAG-3 is provided in GenBank as accession number NP_002277.4. LAG-3 is a member of the immunoglobulin (Ig) superfamily. LAG-3 is a 503-amino acid type-1 transmembrane protein with four extracellular Ig-like domains D1 to D4 and is expressed on activated T cells, natural killer cells, B cells, plasmacytoid dendritic cells, and regulatory T cells. The LAG-3 receptor binds to MHC class II molecules present on antigen presenting cells (APCs).

As used herein, the term “T cell co-inhibitor” refers to a ligand and/or receptor which modulates the immune response via T cell activation or suppression. The term “T cell co-inhibitor”, also known as T cell co-signaling molecule, includes, but is not limited to, programmed death-1 (PD-1), cytotoxic T-lymphocyte antigen-4 (CTLA-4), B and T lymphocyte attenuator (BTLA), CD-28, 2B4, LY108, T cell immunoglobulin and mucin 3(TIM3), T cell immunoreceptor with immunoglobulin and ITIM (TIGIT; also known as VSIG9), leucocyte associated immunoglobulin-like receptor 1 (LAIR1; also known as CD305), inducible T cell costimulator (ICOS; also known as CD278), V-domain Ig suppressor of T cell activation (VISTA) and CD160.

LAG-3 inhibitors include antibodies and antigen-binding fragments thereof and other substances (e.g., peptides and small molecules) that specifically bind to LAG-3 and antagonize one or more biological activities of LAG-3. Molecules that specifically bind to LAG-3 may be referred to as “anti-LAG-3”.

In an embodiment of the disclosure, the LAG-3 inhibitor is an antibody or antigen-binding fragment thereof as set forth in U.S. 20170101472.

According to certain embodiments, the antibodies used in the methods of the present disclosure specifically bind LAG-3. The term “specifically binds,” or the like, means that an antibody or antigen-binding fragment thereof forms a complex with an antigen that is relatively stable under physiologic conditions. Methods for determining whether an antibody specifically binds to an antigen are well known in the art and include, for example, equilibrium dialysis, surface plasmon resonance, and the like. For example, an antibody that “specifically binds” LAG-3, as used in the context of the present disclosure, includes antibodies that bind LAG-3 or portion thereof with a K_{D} of less than about 500 nM, less than about 300 nM, less than about 200 nM, less than about 100 nM, less than about 90 nM, less than about 80 nM, less than about 70 nM, less than about 60 nM, less than about 50 nM, less than about 40 nM, less than about 30 nM, less than about 20 nM, less than about 10 nM, less than about 5 nM, less than about 4 nM, less than about 3 nM, less than about 2 nM, less than about 1 nM or less than about 0.5 nM, as measured in a surface plasmon resonance assay. An isolated antibody that specifically binds human LAG-3 may, however, have cross-reactivity to other antigens, such as LAG-3 molecules from other (non-human) species.

According to certain exemplary embodiments of the present disclosure, the anti-LAG-3 antibody, or antigen-binding fragment thereof comprises a heavy chain variable region (HCVR), light chain variable region (LCVR), and/or complementarity determining regions (CDRs) comprising any of the amino acid sequences of the anti-LAG-3 antibodies as set forth in U.S. 20170101472.

In certain exemplary embodiments, the anti-LAG-3 antibody or antigen-binding fragment thereof that can be used in the context of the methods of the present disclosure comprises the heavy chain complementarity determining regions (HCDRs) of a heavy chain variable region (HCVR) comprising the amino acid sequence of SEQ ID NO: 11 and the light chain complementarity determining regions (LCDRs) of a light chain variable region (LCVR) comprising the amino acid sequence of SEQ ID NO: 12. According to certain embodiments, the anti-LAG-3 antibody or antigen-binding fragment thereof comprises three HCDRs (HCDR1, HCDR2 and HCDR3) and three LCDRs (LCDR1, LCDR2 and LCDR3), wherein the HCDR1 comprises the amino acid sequence of SEQ ID NO: 13; the HCDR2 comprises the amino acid sequence of SEQ ID NO: 14; the HCDR3 comprises the amino acid sequence of SEQ ID NO: 15; the LCDR1 comprises the amino acid sequence of SEQ ID NO: 16; the LCDR2 comprises the amino acid sequence of SEQ ID NO: 17; and the LCDR3 comprises the amino acid sequence of SEQ ID NO: 18. In yet other embodiments, the anti-LAG-3 antibody or antigen-binding fragment thereof comprises an HCVR comprising SEQ ID NO: 11 and an LCVR comprising SEQ ID NO: 12. In certain embodiments, the methods of the present disclosure comprise the use of an anti-LAG-3 antibody, wherein the antibody comprises a heavy chain comprising the amino acid sequence of SEQ ID NO: 19. In some embodiments, the anti-LAG-3 antibody comprises a light chain comprising the amino acid sequence of SEQ ID NO: 20. An exemplary antibody comprising a heavy chain variable region comprising the amino acid sequence of SEQ ID NO: 11 and a light chain variable region comprising the amino acid sequence of SEQ ID NO: 12 is the fully human anti-LAG-3 antibody known as REGN3767 (fianlimab).

According to certain exemplary embodiments, the methods of the present disclosure comprise the use of REGN3767, or a bioequivalent thereof. The term “bioequivalent”, as used herein, refers to anti-LAG-3 antibodies or LAG-3-binding proteins or fragments thereof that are pharmaceutical equivalents or pharmaceutical alternatives whose rate and/or extent of absorption do not show a significant difference with that of REGN3767 when administered at the same molar dose under similar experimental conditions, either single dose or multiple dose. In the context of the disclosure, the term refers to antigen-binding proteins that bind to LAG-3 which do not have clinically meaningful differences with REGN3767 in their safety, purity and/or potency.

Other anti-LAG-3 antibodies that can be used in the context of the methods of the present disclosure include, e.g., the antibodies referred to and known in the art as relatlimab (U.S. 20110150892), LAG525 (WO2017/037203), GSK2831781 (U.S. 2016/0017037), Sym022 (WO2018/069500), INCAGN02385 (U.S. 20180127499) or any of the anti-LAG-3 antibodies as set forth in U.S. Pat. Nos. 5,976,877, 6,143,273, 6,197,524, 8,551,481, 20110070238, 20110150892, 20130095114, 20140093511, 20140127226, 20140286935, and in WO95/30750, WO97/03695, WO98/58059, WO2004/078928, WO2008/132601, WO2010/019570, WO2014/008218, EP0510079B1, EP0758383B1, EP0843557B1, EP0977856B1, EP1897548B2, EP2142210A1, and EP2320940B1.

### Methods for Treating Melanoma or Inhibiting Melanoma Tumor Growth

The present disclosure includes methods for treating, ameliorating or reducing the severity of at least one symptom or indication, or inhibiting the growth of a melanoma in a subject. The methods according to this aspect comprise administering an antibody or antigen-binding fragment thereof that specifically binds PD-1 in combination with an antibody or antigen-binding fragment thereof that specifically binds LAG-3 to a subject in need thereof. In some aspects, the methods comprise administering a therapeutically effective amount of an antibody or antigen-binding fragment thereof that specifically binds PD-1 in combination with a therapeutically effective amount of an antibody or antigen-binding fragment thereof that specifically binds LAG-3 to a subject in need thereof. As used herein, the terms “treat”, “treating”, or the like, mean to alleviate symptoms, eliminate the causation of symptoms either on a temporary or permanent basis, to delay or inhibit melanoma growth, to reduce melanoma cell load or tumor burden, to promote melanoma regression, to cause melanoma shrinkage, necrosis and/or disappearance, to prevent melanoma recurrence, and/or to increase duration of survival of the subject.

As used herein, the expression “a subject in need thereof” means a human or non-human mammal that exhibits one or more symptoms or indications of melanoma, and/or who has been diagnosed with melanoma, and who needs treatment for the same. In many embodiments, the term “subject” may be interchangeably used with the term “patient”. For example, a human subject may be diagnosed with a primary or a metastatic melanoma and/or with one or more symptoms or indications including, but not limited to, enlarged lymph node(s), swollen abdomen, chest pain/pressure, unexplained weight loss, fever, night sweats, persistent fatigue, loss of appetite, enlargement of spleen, itching. In specific embodiments, the expression includes human subjects that have and need treatment for unresectable locally advanced melanoma or metastatic melanoma. In some embodiments, the human subjects have not received a previous systemic treatment for advanced disease. In specific embodiments, the expression includes human subjects that have and need treatment for completely resected high-risk melanoma in the adjuvant setting. In certain embodiments, the expression “a subject in need thereof” includes patients with a melanoma that is resistant to or refractory to or is inadequately controlled by prior therapy (e.g., treatment with a conventional anti-cancer agent or therapy such as radiation, chemotherapy or surgery, or treatment with an anti-cancer biologic). For example, the expression includes subjects who have been treated with a PD-1 or PD-L1 inhibitor (e.g., an anti-PD-1 antibody). The expression also includes subjects with a melanoma for which conventional anti-cancer therapy is inadvisable, for example, due to toxic side effects. For example, the expression includes patients who have received one or more cycles of chemotherapy with toxic side effects. In certain embodiments, the expression “a subject in need thereof” includes patients with a melanoma which has been treated but which has subsequently relapsed or metastasized. For example, patients with melanoma that may have received treatment with one or more anti-cancer agents leading to melanoma regression; however, subsequently have relapsed with melanoma resistant to the one or more anti-cancer agents (e.g., chemotherapy-resistant melanoma) are treated with the methods provided herein.

In some instances, a patient having unresectable locally advanced melanoma or metastatic melanoma is further selected as having one or more of the following criteria:
(i) at least 12 years of age on the date of providing informed consent;
(ii) either stage IIC, III, or stage IV per AJCC 8th edition (Amin, 2017) and have histologically confirmed melanoma that is completely surgically resected in order to be eligible;
(iii) patients with stage IIIA disease must have at least 1 lymph node micrometastasis measuring >1 mm in greatest diameter;
(iv) Stage IIC melanoma confirmed by a pathologically negative SLNB specimen and no evidence of regional or distant metastasis;
(v) complete surgical resection performed within 12 weeks prior to treatment, and enrollment may occur only after satisfactory wound healing from the surgery;
(vi) disease-free status documented by a complete physical examination and imaging studies within 4 weeks prior to treatment;
(vii) for patients with abnormal or suspicious findings in the screening scans, residual malignancy and/or metastatic disease must be excluded by ¹⁸F-FDG PET-CT and/or biopsy; and
(viii) patients must not have received systemic anti-cancer therapy or radiation therapy for melanoma in the previous 5 years, with the exception of stage IV M1d patients who may have had RT to the CNS following radical resection. Adjuvant radiation therapy to the primary site and/or to the nodal bed following radical surgical resection is allowed where it is considered institutional standard of care.

In some instances, a having a completely resected high-risk melanoma in the adjuvant setting is further selected as having one or more of the following criteria:
(1) ≥12 years of age;
(2) Stage IIc, III or IV (all M-stages) and histologically confirmed melanoma, completely resected <12 weeks prior to treatment;
(3) no prior systemic anti-cancer therapy or radiation therapy for melanoma in the previous 5 years;
(4) no evidence of metastatic disease on staging investigations; and
(5) an Eastern Cooperative Oncology Group performance status (PS) of 0 or 1 (for adult pts), Karnofsky PS>70 (pts>16 years) or Lansky PS>70 (pts<16 years).

The expression “a subject in need thereof” also includes subjects who are at risk of developing a melanoma, e.g., persons with a family history of melanoma, persons with a past melanoma occurrence, or persons with a compromised immune system. In some aspects, the subject is resistant or inadequately responsive to, or relapsed after prior therapy.

In certain embodiments, the methods provided herein may be used to treat patients that show elevated levels of one or more cancer-associated biomarkers (e.g., PD-L1, or LAG-3). For example, the methods of the present invention comprise administering a therapeutically effective amount of an anti-LAG-3 antibody in combination with an anti-PD-1 antibody to a patient with an elevated level of LAG-3 and/or PD-L1. In one embodiment, the present methods are used in patients with melanoma that are selected on the basis of LAG-3 expression in cancer tissue, wherein the cancer tissue comprises melanoma cells and tumor-infiltrating immune cells. In certain embodiments, the present methods are used to treat patients with a melanoma wherein the patients are selected on the basis of ≥1% LAG-3 expression in cancer tissue and/or immune cells. In one embodiment, the present methods are used in patients with melanoma that are selected on the basis of LAG-3 expression in cancer tissue, wherein the cancer tissue comprises melanoma cells and tumor-infiltrating immune cells. In certain embodiments, the present methods are used to treat patients with a melanoma wherein the patients are selected on the basis of ≥1% LAG-3 expression in cancer tissue and/or immune cells. Methods to determine LAG-3 or PD-L1 expression in cancer tissue and/or tumor-associated immune cells are well-known in the art. In certain embodiments, the expression of LAG-3 in tumor tissue is determined by any assay known in the art, for example, by an ELISA assay or by an immunohistochemistry (IHC) assay (e.g., as described in He et al 2017, J. Thoracic Oncol. 12: 814-823; WO2016124558 or WO2016191751). In certain embodiments, the expression of LAG-3 or PD-L1 is determined by quantitating RNA expression, for example, by in situ hybridization or by RT-PCR. In certain embodiments, the expression of LAG-3 is determined by imaging with a labeled anti-LAG-3 antibody, for example, by immuno-positron emission tomography or iPET [See, e.g., The Oncologist, 12: 1379 (2007); Journal of Nuclear Medicine, 52(8): 1171 (2011); US Patent Application Publication 2018/0228926]. In certain embodiments, the expression of PD-L1 is determined by imaging with a labeled anti-PD-L1 antibody, for example, by immuno-positron emission tomography or iPET (US Patent Application Publication 2018/0161464).

In certain embodiments, the methods provided herein are used in a subject with a cancer. The terms “tumor”, “cancer” and “malignancy” are interchangeably used herein.

In certain embodiments, the cancer or tumor is a melanoma. Reference to “tumor” or “cancer” throughout is inclusive of melanoma, e.g., the tumor or cancer is melanoma. In some aspects, the melanoma is unresectable locally advanced melanoma. In some aspects, the melanoma is metastatic melanoma. In some aspects, the patient has not received a previous systemic treatment for advanced disease. In other aspects, the melanoma is a completely resected high-risk melanoma. In some aspects, the melanoma is a completely resected high-risk melanoma in an adjuvant setting.

According to certain embodiments, the present disclosure includes methods for treating, or delaying or inhibiting the growth of a melanoma tumor. In certain embodiments, this includes methods to promote melanoma regression. In certain embodiments, this includes methods to reduce tumor cell load or to reduce tumor burden. In certain embodiments, the present disclosure includes methods to prevent melanoma recurrence. The methods, according to this aspect, comprise sequentially administering a therapeutically effective amount of an anti-PD-1 antibody in combination with anti-LAG-3 antibody to a subject in need thereof, wherein each antibody is administered to the subject in multiple doses, e.g., as part of a specific therapeutic dosing regimen. For example, the therapeutic dosing regimen may comprise administering one or more doses of an anti-PD-1 antibody to the subject at a frequency of about once a day, once every two days, once every three days, once every four days, once every five days, once every six days, once a week, once every two weeks, once every three weeks, once every four weeks, once a month, once every six weeks, once every two months, once every three months, once every four months, or less frequently. In certain embodiments, the one or more doses of anti-PD-1 antibody are administered in combination with one or more doses of a therapeutically effective amount of anti-LAG-3 antibody, wherein the one or more doses of the anti-LAG-3 antibody are administered to the subject at a frequency of about once a day, once every two days, once every three days, once every four days, once every five days, once every six days, once a week, once every two weeks, once every three weeks, once every four weeks, once a month, once every six weeks, once every two months, once every three months, once every four months, or less frequently.

In certain embodiments, the present disclosure includes methods to inhibit, retard or stop melanoma metastasis or infiltration into peripheral organs. The methods, according to this aspect, comprise administering a therapeutically effective amount of an anti-PD-1 antibody to a subject in need thereof. In certain embodiments, the anti-PD-1 antibody is administered in combination with an anti-LAG-3 antibody.

In specific embodiments, the present disclosure provides methods for increased anti-tumor efficacy or increased melanoma inhibition. In certain embodiments, the methods provide for increased melanoma inhibition, e.g., by about 20%, more than 20%, more than 30%, more than 40% more than 50%, more than 60%, more than 70% or more than 80% as compared to a subject administered either antibody as a monotherapy.

The methods provided herein, according to certain embodiments, comprise administering to a subject with melanoma a therapeutically effective amount of an anti-PD-1 antibody prior to, concurrent with, or after administering a therapeutically effective amount of anti-LAG-3 antibody. In some aspects, the anti-PD-1 antibody may be administered about 1 day, more than 1 day, more than 2 days, more than 3 days, more than 4 days, more than 5 days, more than 6 days, more than 7 days, or more than 8 days prior to the anti-LAG-3 antibody. In some aspects, the anti-PD-1 antibody and anti-LAG-3 antibody are administered concurrently, or within 30 minutes, or within 60 minutes, or within 2 hours, or within 3 hours, or within a day of each other.

In certain embodiments, the methods provided herein comprise administering a therapeutically effective amount of an anti-PD-1 antibody to a subject with melanoma. In specific embodiments, the melanoma is indolent or aggressive. In certain embodiments, the subject is not responsive to prior therapy or has relapsed after prior therapy. Prior therapy can include surgery, radiation, and/or chemotherapy, or treatment with a PD-1 inhibitor, a PD-L1 inhibitor, and/or any other anti-cancer biologic.

In certain embodiments, the methods of the present disclosure comprise administering an anti-PD-1 antibody in combination with an anti-LAG-3 antibody to a subject in need thereof as a “first line” treatment (e.g., initial treatment). In other embodiments, an anti-PD-1 antibody in combination with anti-LAG-3 antibody is administered as a “second line” treatment (e.g., after prior therapy). For example, an anti-PD-1 antibody in combination with anti-LAG-3 antibody is administered as a “second line” treatment to a subject that has relapsed after prior therapy with, e.g., chemotherapy or rituximab.

In certain embodiments, the methods of the present disclosure are used to treat a patient with an MRD-positive disease. Minimum residual disease (MRD) refers to small numbers of cancer cells that remain in the patient during or after treatment, wherein the patient may or may not show symptoms or signs of the disease. Such residual cancer cells, if not eliminated, frequently lead to relapse of the disease. The present disclosure includes methods to inhibit and/or eliminate residual cancer cells in a patient upon MRD testing. MRD may be assayed according to methods known in the art (e.g., MRD flow cytometry). The methods, according to this aspect of the disclosure, comprise administering an anti-PD-1 antibody in combination with an anti-LAG-3 antibody to a subject in need thereof.

The methods provided herein, according to certain embodiments, comprise administering to a subject a therapeutically effective amount of each of an anti-PD-1 antibody and an anti-LAG-3 antibody in combination with a third therapeutic agent. The third therapeutic agent may be an agent selected from the group consisting of, e.g., radiation, chemotherapy, surgery, a cancer vaccine, CART, a PD-L1 inhibitor (e.g., an anti-PD-L1 antibody), an CD3 inhibitor, a CD20 inhibitor, a CTLA-4 inhibitor, a CD38 inhibitor, a TIM3 inhibitor, a BTLA inhibitor, a TIGIT inhibitor, a CD47 inhibitor, an indoleamine-2,3-dioxygenase (IDO) inhibitor, a vascular endothelial growth factor (VEGF) antagonist, an Ang2 inhibitor, a transforming growth factor beta (TGFβ) inhibitor, an epidermal growth factor receptor (EGFR) inhibitor, an antibody to a tumor-specific antigen [e.g., CA9, CA125, melanoma-associated antigen 3 (MAGE3), carcinoembryonic antigen (CEA)], Bacillus Calmette-Guerin vaccine, granulocyte-macrophage colony-stimulating factor, an oncolytic virus, a cytotoxin, a CD28 agonist, a GITR agonist, a 4-1BB agonist, CD20xCD3 bispecific antibody (e.g., REGN1979), MUC16xCD3 bispecific antibody, vimentin, tumor-M2-PK, prostate-specific antigen (PSA), mucin-1, MART-1, and CA19-9), a vaccine (e.g., Bacillus Calmette-Guerin), granulocyte-macrophage colony-stimulating factor, a cytotoxin, a chemotherapeutic agent, an IL-6R inhibitor, an IL-4R inhibitor, an IL-10 inhibitor, a cytokine such as IL-2, IL-7, IL-12, IL-21, and IL-15, an anti-inflammatory drug such as corticosteroids, and non-steroidal anti-inflammatory drugs.

In certain embodiments, the antibodies may be administered in combination with therapy including a chemotherapeutic agent, radiation, or surgery. As used herein, the phrase “in combination with” means that the antibodies are administered to the subject at the same time as, just before, or just after administration of the third therapeutic agent. In certain embodiments, the third therapeutic agent is administered as a co-formulation with the antibodies. In a related embodiment, the present disclosure includes methods comprising administering a therapeutically effective amount of an anti-PD-1 antibody in combination with an anti-LAG-3 antibody to a subject who is on a background anti-cancer therapeutic regimen. The background anti-cancer therapeutic regimen may comprise a course of administration of, e.g., a chemotherapeutic agent, or radiation. The anti-PD-1 antibody in combination with the anti-LAG-3 antibody may be added on top of the background anti-cancer therapeutic regimen. In some embodiments, the antibodies are added as part of a “background step-down” scheme, wherein the background anti-cancer therapy is gradually withdrawn from the subject over time (e.g., in a stepwise fashion) while the antibodies are administered to the subject at a constant dose, or at an increasing dose, or at a decreasing dose, over time.

In certain embodiments, the methods of the present disclosure comprise administering to a subject in need thereof a therapeutically effective amount of an anti-PD-1 antibody in combination with a therapeutically effective amount of an anti-LAG-3 antibody, wherein administration of the antibodies leads to increased inhibition of melanoma growth. In certain embodiments, melanoma growth is inhibited by at least about 10%, about 20%, about 30%, about 40%, about 50%, about 60%, about 70% or about 80% as compared to an untreated subject or a subject administered with either antibody as monotherapy. In certain embodiments, the administration of an anti-PD-1 antibody and/or anti-LAG-3 antibody to a subject leads to increased melanoma regression, tumor shrinkage and/or disappearance. In certain embodiments, the administration of an anti-PD-1 antibody and/or an anti-LAG-3 antibody leads to delay in melanoma growth and development, e.g., melanoma growth may be delayed by about 3 days, more than 3 days, about 7 days, more than 7 days, at least 10 days, more than 15 days, more than 1 month, more than 3 months, more than 6 months, more than 1 year, more than 2 years, or more than 3 years as compared to an untreated subject or a subject treated with either antibody as monotherapy. In certain embodiments, administration of an anti-PD-1 antibody in combination with an anti-LAG-3 antibody prevents melanoma recurrence and/or increases duration of survival of the subject, e.g., increases duration of survival by more than 15 days, more than 1 month, more than 3 months, more than 6 months, more than 12 months, more than 18 months, more than 24 months, more than 36 months, or more than 48 months than an untreated subject or a subject which is administered either antibody as monotherapy. In certain embodiments, administration of the antibodies in combination increases progression-free survival or overall survival. In certain embodiments, administration of an anti-PD-1 antibody in combination with an anti-LAG-3 antibody increases response and duration of response in a subject, e.g., by more than 2%, more than 3%, more than 4%, more than 5%, more than 6%, more than 7%, more than 8%, more than 9%, more than 10%, more than 20%, more than 30%, more than 40% or more than 50% over an untreated subject or a subject which has received either antibody as monotherapy. In certain embodiments, administration of an anti-PD-1 antibody and/or an anti-LAG-3 antibody to a subject with melanoma leads to complete disappearance of all evidence of melanoma cells (“complete response”). In certain embodiments, administration of an anti-PD-1 antibody and/or an anti-LAG-3 antibody to a subject with a melanoma leads to at least 30% or more decrease in melanoma cells or tumor size (“partial response”). In certain embodiments, administration of an anti-PD-1 antibody and/or an anti-LAG-3 antibody to a subject with melanoma leads to complete or partial disappearance of melanoma cells/lesions including new measurable lesions. Melanoma tumor reduction can be measured by any of the methods known in the art, e.g., X-rays, positron emission tomography (PET), computed tomography (CT), magnetic resonance imaging (MRI), cytology, histology, or molecular genetic analyses. In some aspects, administration of an anti-PD-1 antibody in combination with an anti-LAG-3 antibody to a patient population unexpectedly results in more patients responding to treatment, unexpectedly results in patient responses to treatment that are longer even without more patients responding, and/or the patients that do respond to therapy have deeper responses.

In certain embodiments, the combination of administered antibodies is safe and well-tolerated by a patient wherein there is no increase or a tolerable increase in an adverse side effect as compared to a patient administered with either antibody as monotherapy.

### Combination Therapies

The methods of the present disclosure, according to certain embodiments, comprise administering to the subject an anti-LAG-3 antibody in combination with an anti-PD-1 antibody. In certain embodiments, the methods of the present disclosure comprise administering the antibodies for additive or synergistic activity to treat melanoma. As used herein, the expression “in combination with” means that the anti-LAG-3 antibody is administered before, after, or concurrent with the anti-PD-1 antibody. The term “in combination with” also includes sequential or concomitant administration of anti-PD-1 antibody and an anti-LAG-3 antibody. For example, when administered “before” the anti-LAG-3 antibody, the anti-PD-1 antibody may be administered more than 150 hours, about 150 hours, about 100 hours, about 72 hours, about 60 hours, about 48 hours, about 36 hours, about 24 hours, about 12 hours, about 10 hours, about 8 hours, about 6 hours, about 4 hours, about 2 hours, about 1 hour, about 30 minutes, about 15 minutes or about 10 minutes prior to the administration of the anti-LAG-3 antibody. When administered “after” the anti-LAG-3 antibody, the anti-PD-1 antibody may be administered about 10 minutes, about 15 minutes, about 30 minutes, about 1 hour, about 2 hours, about 4 hours, about 6 hours, about 8 hours, about 10 hours, about 12 hours, about 24 hours, about 36 hours, about 48 hours, about 60 hours, about 72 hours, or more than 72 hours after the administration of the anti-LAG-3 antibody. Administration “concurrent” with the anti-LAG-3 antibody means that the anti-PD-1 antibody is administered to the subject in a separate dosage form within less than 5 minutes (before, after, or at the same time) of administration of the anti-LAG-3 antibody, e.g. within 5 minutes of completion of anti-LAG-3 antibody infusion, or administered to the subject as a single combined dosage formulation comprising both the anti-PD-1 antibody and the anti-LAG-3 antibody. In some aspects, the anti-PD-1 antibody is administered the same day as the anti-LAG-3 antibody. In some aspects, the anti-PD-1 antibody and the anti-LAG-3 antibody are administered in a separate dosage form but within 8 hours of each other, for example, within 6 hours, or within 5 hours, or within 4 hours, or within 3 hours, or within 2 hours, or within 60 minutes of each other.

In certain embodiments, the methods provided herein comprise administration of a third therapeutic agent wherein the third therapeutic agent is an anti-cancer drug. As used herein, “anti-cancer drug” means any agent useful to treat cancer including, but not limited to, cytotoxins and agents such as antimetabolites, alkylating agents, anthracyclines, antibiotics, antimitotic agents, procarbazine, hydroxyurea, asparaginase, corticosteroids, mytotane (O,P′-(DDD)), biologics (e.g., antibodies and interferons) and radioactive agents. As used herein, “a cytotoxin or cytotoxic agent”, also refers to a chemotherapeutic agent and means any agent that is detrimental to cells. Examples include Taxol® (paclitaxel), temozolamide, cytochalasin B, gramicidin D, ethidium bromide, emetine, cisplatin, mitomycin, etoposide, tenoposide, vincristine, vinbiastine, coichicin, doxorubicin, daunorubicin, dihydroxy anthracin dione, mitoxantrone, mithramycin, actinomycin D, 1-dehydrotestosterone, glucocorticoids, procaine, tetracaine, lidocaine, propranolol, and puromycin and analogs or homologs thereof. In certain embodiments, the methods provided herein comprise administration of a third therapeutic agent selected from the group consisting of radiation, surgery, a cancer vaccine, a PD-L1 inhibitor (e.g., an anti-PD-L1 antibody), a CD20 inhibitor, a CD3 inhibitor, a CTLA-4 inhibitor (e.g., ipilimumab), a CD38 inhibitor, a TIM3 inhibitor, a BTLA inhibitor, a TIGIT inhibitor, a CD47 inhibitor, an antagonist of another T-cell co-inhibitor or ligand (e.g., an antibody to CD-28, 2B4, LY108, LAIR1, ICOS, CD160 or VISTA), an indoleamine-2,3-dioxygenase (IDO) inhibitor, a vascular endothelial growth factor (VEGF) antagonist [e.g., a “VEGF-Trap” such as aflibercept or other VEGF-inhibiting fusion protein as set forth in U.S. Pat. No. 7,087,411, or an anti-VEGF antibody or antigen binding fragment thereof (e.g., bevacizumab, or ranibizumab) or a small molecule kinase inhibitor of VEGF receptor (e.g., sunitinib, sorafenib, or pazopanib)], an Ang2 inhibitor (e.g., nesvacumab), a transforming growth factor beta (TGFβ) inhibitor, an epidermal growth factor receptor (EGFR) inhibitor (e.g., erlotinib, cetuximab), an agonist to a co-stimulatory receptor (e.g., an agonist to glucocorticoid-induced TNFR-related protein), an antibody to a tumor-specific antigen [e.g., CA9, CA125, melanoma-associated antigen 3 (MAGE3), carcinoembryonic antigen (CEA)], a CD28 agonist, a GITR agonist, a 4-1BB agonist, CD20xCD3 bispecific antibody (e.g., REGN1979), MUC16xCD3 bispecific antibody, vimentin, tumor-M2-PK, prostate-specific antigen (PSA), mucin-1, MART-1, and CA19-9), a vaccine (e.g., Bacillus Calmette-Guerin, a cancer vaccine), an adjuvant to increase antigen presentation (e.g., granulocyte-macrophage colony-stimulating factor), an oncolytic virus, a cytotoxin, a chemotherapeutic agent (e.g., dacarbazine, temozolomide, cyclophosphamide, docetaxel, doxorubicin, daunorubicin, cisplatin, carboplatin, gemcitabine, methotrexate, mitoxantrone, oxaliplatin, paclitaxel, and vincristine), radiotherapy, an IL-6R inhibitor (e.g., sarilumab), an IL-4R inhibitor (e.g., dupilumab), an IL-10 inhibitor, a cytokine such as IL-2, IL-7, IL-12, IL-21, and IL-15, an antibody-drug conjugate (ADC) (e.g., anti-CD19-DM4 ADC, and anti-DS6-DM4 ADC), chimeric antigen receptor T cells (e.g., CD19-targeted T cells) or other cell therapies, and an anti-inflammatory drug (e.g., corticosteroids, and non-steroidal anti-inflammatory drugs).

In certain embodiments, the methods provided herein comprise administering an anti-PD-1 antibody and an anti-LAG-3 antibody in combination with radiation therapy/chemotherapy to generate long-term durable anti-tumor responses and/or enhance survival of patients with melanoma.

In some embodiments, the methods of the disclosure comprise administering radiation therapy prior to, concomitantly or after administering an anti-PD-1 antibody and an anti-LAG-3 antibody to a melanoma patient. For example, radiation therapy may be administered in one or more doses to melanoma lesions after administration of one or more doses of the antibodies. In some embodiments, radiation therapy may be administered locally to a melanoma lesion to enhance the local immunogenicity of a patient's melanoma (adjuvinating radiation) and/or to kill melanoma cells (ablative radiation) before or after systemic administration of an anti-PD-1 antibody and/or an anti-LAG-3 antibody, i.e., in an adjuvant setting. In certain embodiments, the antibodies may be administered in combination with radiation therapy and a chemotherapeutic agent (e.g., temozolomide or cyclophosphamide) or a VEGF antagonist (e.g., aflibercept).

### Pharmaceutical Compositions and Administration

Provided herein are methods which comprise administering an anti-PD-1 antibody in combination with an anti-LAG-3 antibody to a subject wherein the antibodies are contained within separate or combined (single) pharmaceutical composition. The pharmaceutical compositions of the disclosure may be formulated with suitable carriers, excipients, and other agents that provide suitable transfer, delivery, tolerance, and the like. A multitude of appropriate formulations can be found in the formulary known to all pharmaceutical chemists: Remington's Pharmaceutical Sciences, Mack Publishing Company, Easton, Pa. These formulations include, for example, powders, pastes, ointments, jellies, waxes, oils, lipids, lipid (cationic or anionic) containing vesicles (such as LIPOFECTIN™), DNA conjugates, anhydrous absorption pastes, oil-in-water and water-in-oil emulsions, emulsions carbowax (polyethylene glycols of various molecular weights), semi-solid gels, and semi-solid mixtures containing carbowax. See also Powell et al. “Compendium of excipients for parenteral formulations” PDA (1998) J Pharm Sci Technol 52:238-311.

Various delivery systems are known and can be used to administer the pharmaceutical composition of the disclosure, e.g., encapsulation in liposomes, microparticles, microcapsules, recombinant cells capable of expressing the mutant viruses, receptor mediated endocytosis (see, e.g., Wu et al., 1987, J. Biol. Chem. 262: 4429-4432). Methods of administration include, but are not limited to, intradermal, intramuscular, intraperitoneal, intravenous, subcutaneous, intranasal, epidural, and oral routes. The composition may be administered by any convenient route, for example by infusion or bolus injection, by absorption through epithelial or mucocutaneous linings (e.g., oral mucosa, rectal and intestinal mucosa, etc.) and may be administered together with other biologically active agents.

A pharmaceutical composition of the present disclosure can be delivered subcutaneously or intravenously with a standard needle and syringe. In one embodiment, the syringe is a pre-filled syringe. In addition, with respect to subcutaneous delivery, a pen delivery device readily has applications in delivering a pharmaceutical composition of the present disclosure. Such a pen delivery device can be reusable or disposable. A reusable pen delivery device generally utilizes a replaceable cartridge that contains a pharmaceutical composition. Once all of the pharmaceutical composition within the cartridge has been administered and the cartridge is empty, the empty cartridge can readily be discarded and replaced with a new cartridge that contains the pharmaceutical composition. The pen delivery device can then be reused. In a disposable pen delivery device, there is no replaceable cartridge. Rather, the disposable pen delivery device comes prefilled with the pharmaceutical composition held in a reservoir within the device. Once the reservoir is emptied of the pharmaceutical composition, the entire device is discarded.

In certain situations, the pharmaceutical composition can be delivered in a controlled release system. In one embodiment, a pump may be used. In another embodiment, polymeric materials can be used; see, Medical Applications of Controlled Release, Langer and Wise (eds.), 1974, CRC Pres., Boca Raton, Fla. In yet another embodiment, a controlled release system can be placed in proximity of the composition's target, thus requiring only a fraction of the systemic dose (see, e.g., Goodson, 1984, in Medical Applications of Controlled Release, supra, vol. 2, pp. 115-138). Other controlled release systems are discussed in the review by Langer, 1990, Science 249:1527-1533.

The injectable preparations may include dosage forms for intravenous, subcutaneous, intracutaneous and intramuscular injections, drip infusions, etc. These injectable preparations may be prepared by known methods. For example, the injectable preparations may be prepared, e.g., by dissolving, suspending or emulsifying the antibody or its salt described above in a sterile aqueous medium or an oily medium conventionally used for injections. As the aqueous medium for injections, there are, for example, physiological saline, an isotonic solution containing glucose and other auxiliary agents, etc., which may be used in combination with an appropriate solubilizing agent such as an alcohol (e.g., ethanol), a polyalcohol (e.g., propylene glycol, polyethylene glycol), a nonionic surfactant [e.g., polysorbate 80, HCO-50 (polyoxyethylene (50 mol) adduct of hydrogenated castor oil)], etc. As the oily medium, there are employed, e.g., sesame oil, soybean oil, etc., which may be used in combination with a solubilizing agent such as benzyl benzoate, benzyl alcohol, etc. The injection thus prepared is preferably filled in an appropriate ampoule.

Advantageously, the pharmaceutical compositions for oral or parenteral use described above are prepared into dosage forms in a unit dose suited to fit a dose of the active ingredients. Such dosage forms in a unit dose include, for example, tablets, pills, capsules, injections (ampoules), suppositories, etc.

### Administration Regimens

The present disclosure includes methods comprising administering to a subject an anti-PD-1 antibody at a dosing frequency of about four times a week, twice a week, once a week, once every two weeks, once every three weeks, once every four weeks, once every five weeks, once every six weeks, once every eight weeks, once every twelve weeks, or less frequently so long as a therapeutic response is achieved. In certain embodiments, the present disclosure includes methods comprising administering to a subject an anti-LAG-3 antibody at a dosing frequency of about four times a week, twice a week, once a week, once every two weeks, once every three weeks, once every four weeks, once every five weeks, once every six weeks, once every eight weeks, once every twelve weeks, or less frequently so long as a therapeutic response is achieved. In certain embodiments, the methods involve the administration of an anti-PD-1 antibody in combination with an anti-LAG-3 antibody at a dosing frequency of about four times a week, twice a week, once a week, once every two weeks, once every three weeks, once every four weeks, once every five weeks, once every six weeks, once every eight weeks, once every nine weeks, once every twelve weeks, or less frequently so long as a therapeutic response is achieved.

According to certain embodiments of the present disclosure, multiple doses of an anti-PD-1 antibody in combination with an anti-LAG-3 antibody may be administered to a subject over a defined time course. The methods according to this aspect of the disclosure comprise sequentially administering to a subject one or more doses of an anti-PD-1 antibody in combination with one or more doses of an anti-LAG-3 antibody. As used herein, “sequentially administering” means that each dose of the antibody is administered to the subject at a different point in time, e.g., on different days separated by a predetermined interval (e.g., hours, days, weeks, or months). The present disclosure includes methods which comprise sequentially administering to the patient a single initial dose of an anti-PD-1 antibody, followed by one or more secondary doses of the anti-PD-1 antibody, and optionally followed by one or more tertiary doses of the anti-PD-1 antibody. In certain embodiments, the methods further comprise sequentially administering to the patient a single initial dose of an anti-LAG-3 antibody, followed by one or more secondary doses of the anti-LAG-3 antibody, and optionally followed by one or more tertiary doses of the anti-LAG-3 antibody.

According to certain embodiments of the present disclosure, multiple doses of an anti-PD-1 antibody and an anti-LAG-3 antibody may be administered to a subject over a defined time course. The methods according to this aspect of the disclosure comprise sequentially administering to a subject multiple doses of an anti-PD-1 antibody and an anti-LAG-3 antibody. As used herein, “sequentially administering” means that each dose of the anti-PD-1 antibody in combination with the anti-LAG-3 antibody is administered to the subject at a different point in time, e.g., on different days separated by a predetermined interval (e.g., hours, days, weeks, or months).

According to certain embodiments of the present disclosure, multiple doses of an anti-LAG-3 antibody can be administered to a subject for several months or years, once every 3 or 6 weeks, then the subject is administered the anti-PD-1 antibody in combination with the anti-LAG-3 antibody, for several months or years. In some aspects, the anti-LAG-3 antibody dosage is different as a monotherapy versus the combination therapy. In some aspects, the anti-LAG-3 antibody dosage is the same whether administered as a monotherapy or in combination with the anti-PD-1 antibody.

The terms “initial dose,” “secondary doses,” and “tertiary doses,” refer to the temporal sequence of administration. Thus, the “initial dose” is the dose which is administered at the beginning of the treatment regimen (also referred to as the “baseline dose”); the “secondary doses” are the doses which are administered after the initial dose; and the “tertiary doses” are the doses which are administered after the secondary doses. The initial, secondary, and tertiary doses may all contain the same amount of the antibody (anti-PD-1 antibody or anti-LAG-3 antibody). In certain embodiments, however, the amount contained in the initial, secondary and/or tertiary doses varies from one another (e.g., adjusted up or down as appropriate) during the course of treatment. In certain embodiments, one or more (e.g., 1, 2, 3, 4, or 5) doses are administered at the beginning of the treatment regimen as “loading doses” followed by subsequent doses that are administered on a less frequent basis (e.g., “maintenance doses”). For example, an anti-PD-1 antibody may be administered to a patient with melanoma at a loading dose of about 1-20 mg/kg followed by one or more maintenance doses of about 3 mg/kg of the patient's body weight.

In one exemplary embodiment of the present disclosure, each secondary and/or tertiary dose is administered ½ to 14 (e.g., ½, 1, 1½, 2, 2½, 3, 3½, 4, 4½, 5, 5½, 6, 6½, 7, 7½, 8, 8½, 9, 9½, 10, 10½, 11, 11½, 12, 12½, 13, 13½, 14, 14½, or more) weeks after the immediately preceding dose. The phrase “the immediately preceding dose,” as used herein, means, in a sequence of multiple administrations, the dose of anti-PD-1 antibody (and/or anti-LAG-3 antibody) which is administered to a patient prior to the administration of the very next dose in the sequence with no intervening doses.

The methods according some aspects may comprise administering to a patient any number of secondary and/or tertiary doses of an anti-PD-1 antibody (and/or anti-LAG-3 antibody). For example, in certain embodiments, only a single secondary dose is administered to the patient. In other embodiments, two or more (e.g., 2, 3, 4, 5, 6, 7, 8, or more) secondary doses are administered to the patient. Likewise, in certain embodiments, only a single tertiary dose is administered to the patient. In other embodiments, two or more (e.g., 2, 3, 4, 5, 6, 7, 8, or more) tertiary doses are administered to the patient.

In embodiments involving multiple secondary doses, each secondary dose may be administered at the same frequency as the other secondary doses. For example, each secondary dose may be administered to the patient 1 to 2 weeks after the immediately preceding dose. Similarly, in embodiments involving multiple tertiary doses, each tertiary dose may be administered at the same frequency as the other tertiary doses. For example, each tertiary dose may be administered to the patient 2 to 4 weeks after the immediately preceding dose. Alternatively, the frequency at which the secondary and/or tertiary doses are administered to a patient can vary over the course of the treatment regimen. The frequency of administration may also be adjusted during the course of treatment by a physician depending on the needs of the individual patient following clinical examination.

In certain embodiments, one or more doses of an anti-PD-1 antibody and/or an anti-LAG-3 antibody are administered at the beginning of a treatment regimen as “induction doses” on a more frequent basis (twice a week, once a week or once in 2 weeks) followed by subsequent doses (“consolidation doses” or “maintenance doses”) that are administered on a less frequent basis (e.g., once in 4-12 weeks).

In some embodiments, concomitant administration of anti-PD-1 antibody and the anti-LAG-3 antibody which is administered at a separate dosage at a similar or different frequency relative to the anti-PD-1 antibody is contemplated herein. In some embodiments, the anti-LAG-3 antibody is administered before, after or concurrently with the anti-PD-1 antibody. In certain embodiments, the anti-LAG-3 antibody is administered as a single dosage formulation with the anti-PD-1 antibody.

The present disclosure includes methods comprising sequential administration of an anti-PD-1 antibody in combination with an anti-LAG-3 antibody, to a patient to treat a melanoma. In some embodiments, the present methods comprise administering one or more doses of an anti-PD-1 antibody followed by one or more doses of an anti-LAG-3 antibody. In certain embodiments, the present methods comprise administering a single dose of an anti-PD-1 antibody followed by one or more doses of an anti-LAG-3 antibody. In some embodiments, one or more doses of about 0.1 mg/kg to about 20 mg/kg of an anti-PD-1 antibody may be administered followed by one or more doses of about 0.1 mg/kg to about 50 mg/kg of the anti-LAG-3 antibody to inhibit melanoma growth and/or to prevent melanoma recurrence in a subject with melanoma. In some embodiments, the anti-PD-1 antibody is administered at one or more doses followed by one or more doses of the anti-LAG-3 antibody resulting in increased anti-tumor efficacy (e.g., greater inhibition of melanoma growth, increased prevention of melanoma recurrence as compared to an untreated subject or a subject administered with either antibody as monotherapy).

The present disclosure also includes methods comprising sequential administration of an anti-LAG-3 antibody in combination with an anti-PD-1 antibody, to a patient to treat a melanoma. In some embodiments, the present methods comprise administering one or more doses of an anti-LAG-3 antibody followed by one or more doses of an anti-PD-1 antibody. In certain embodiments, the present methods comprise administering a single dose of an anti-LAG-3 antibody followed by one or more doses of an anti-PD-1 antibody. In some embodiments, one or more doses of about 0.1 mg/kg to about 50 mg/kg of an anti-LAG-3 antibody may be administered followed by one or more doses of about 0.1 mg/kg to about 20 mg/kg of the anti-PD-1 antibody to inhibit tumor growth and/or to prevent melanoma recurrence in a subject with melanoma. In some embodiments, one or more doses of about 50 mg to about 8000 mg of an anti-LAG-3 antibody may be administered followed by one or more doses of about 50 mg to about 1500 mg of the anti-PD-1 antibody to inhibit melanoma growth and/or to prevent melanoma recurrence in a subject with melanoma. In some embodiments, the anti-LAG-3 antibody is administered at one or more doses followed by one or more doses of the anti-PD-1 antibody resulting in increased anti-tumor efficacy (e.g., greater inhibition of melanoma growth, increased prevention of melanoma recurrence as compared to an untreated subject or a subject administered with either antibody as monotherapy).

### Dosage

The amount of anti-PD-1 antibody and/or anti-LAG-3 antibody administered to a subject according to the methods of the present disclosure is, generally, a therapeutically effective amount. As used herein, the phrase “therapeutically effective amount” means an amount of antibody (anti-PD-1 antibody or anti-LAG-3 antibody) that results in, or has the therapeutic effect of, one or more of: (a) a reduction in the severity or duration of a symptom of melanoma; (b) inhibition of melanoma growth, or an increase in melanoma cell necrosis, melanoma tumor shrinkage and/or melanoma tumor disappearance; (c) delay in melanoma growth and development; (d) inhibit or retard or stop melanoma metastasis; (e) prevention of recurrence of melanoma growth; (f) increase in survival of a subject with a melanoma; and/or (g) a reduction in the use or need for conventional anti-cancer therapy (e.g., reduced or eliminated use of chemotherapeutic or cytotoxic agents) as compared to an untreated subject or a subject administered with either antibody as monotherapy.

In the case of an anti-PD-1 antibody, a therapeutically effective amount can be from about 0.05 mg to about 1500 mg, e.g., about 0.05 mg, about 0.1 mg, about 1.0 mg, about 1.5 mg, about 2.0 mg, about 10 mg, about 20 mg, about 30 mg, about 40 mg, about 50 mg, about 60 mg, about 70 mg, about 80 mg, about 90 mg, about 100 mg, about 110 mg, about 120 mg, about 130 mg, about 140 mg, about 150 mg, about 160 mg, about 170 mg, about 180 mg, about 190 mg, about 200 mg, about 210 mg, about 220 mg, about 230 mg, about 240 mg, about 250 mg, about 260 mg, about 270 mg, about 280 mg, about 290 mg, about 300 mg, about 310 mg, about 320 mg, about 330 mg, about 340 mg, about 350 mg, about 360 mg, about 370 mg, about 380 mg, about 390 mg, about 400 mg, about 410 mg, about 420 mg, about 430 mg, about 440 mg, about 450 mg, about 460 mg, about 470 mg, about 480 mg, about 490 mg, about 500 mg, about 510 mg, about 520 mg, about 530 mg, about 540 mg, about 550 mg, about 560 mg, about 570 mg, about 580 mg, about 590 mg, about 600 mg, about 700 mg, about 800 mg, about 900 mg, about 1000 mg, about 1050 mg, about 1100 mg, about 1200 mg, about 1300 mg, about 1400 mg, or about 1500 mg, of the anti-PD-1 antibody. In certain embodiments, 350 mg of an anti-PD-1 antibody is administered. In certain embodiments, 1050 mg of an anti-PD-1 antibody is administered.

In the case of an anti-LAG-3 antibody, a therapeutically effective amount can be from about 10 mg to about 8000 mg, e.g., about 10 mg, about 20 mg, about 50 mg, about 70 mg, about 100 mg, about 120 mg, about 150 mg, about 200 mg, about 250 mg, about 300 mg, about 350 mg, about 400 mg, about 450 mg, about 500 mg, about 550 mg, about 600 mg, about 700 mg, about 800 mg, about 900 mg, about 1000 mg, about 1050 mg, about 1100 mg, about 1500 mg, about 1600 mg, about 1700 mg, about 2000 mg, about 2050 mg, about 2100 mg, about 2200 mg, about 2500 mg, about 2700 mg, about 2800 mg, about 2900 mg, about 3000 mg, about 3200 mg, about 4000 mg, about 5000 mg, about 6000 mg, about 7000 mg, or about 8000 mg of the anti-LAG-3 antibody.

The amount of either anti-PD-1 antibody or anti-LAG-3 antibody contained within the individual doses may be expressed in terms of milligrams of antibody per kilogram of subject body weight (i.e., mg/kg). In certain embodiments, either anti-PD-1 antibody or anti-LAG-3 antibody used in the methods of the present disclosure may be administered to a subject at a dose of about 1 to about 50 mg/kg of subject body weight. For example, anti-PD-1 antibody may be administered at dose of about 0.1 mg/kg to about 20 mg/kg of a patient's body weight. The anti-LAG-3 antibody may be administered at a dose of about 0.1 mg/kg to about 50 mg/kg of a patient's body weight.

### EXAMPLES

The following examples are put forth so as to provide those of ordinary skill in the art with a complete disclosure and description of how to make and use the methods and compositions of the disclosure, and are not intended to limit the scope of what the inventors regard as their disclosure. Efforts have been made to ensure accuracy with respect to numbers used (e.g., amounts, temperature, etc.) but some experimental errors and deviations should be accounted for. Unless indicated otherwise, parts are parts by weight, molecular weight is average molecular weight, temperature is in degrees Centigrade, and pressure is at or near atmospheric. Compositions and methods set forth in the Examples form part of the present disclosure.

Therapeutic monoclonal antibodies (mAbs) that target immune inhibitory receptors (e.g., cytotoxic T lymphocyte-associated protein 4 [CTLA-4], and programmed cell death 1 [PD 1]), have shown impressive clinical activity with an acceptable benefit to risk ratio in several tumor types (Topalian, 2014) (Wolchok, 2013) (Larkin, 2015a) (Baksh, 2015). However, sustained responses were achieved in only a minority of patients, suggesting that combination approaches may be required to overcome tumor immune inhibitory mechanisms (Topalian, 2012).

In the Examples below, this antibody (REGN3767 (INN: fianlimab), an antibody to the LAG-3 receptor that blocks LAG-3/MHC II mediated T-cell inhibition, was tested in combination with cemiplimab (REGN2810), an antibody to the PD-1 receptor that blocks PD 1/programmed death ligand 1 (PD-L1) mediated T-cell inhibition. LIBTAYO® (INN: cemiplimab known as cemiplimab rwlc in the United States) has been approved by several Health Authorities for the treatment of patients with different tumor types.

### Example 1: Phase 3 Clinical Trial of Anti-LAG3 (REGN3767; Fianlimab) and Anti-PD-1 (REGN2810; Cemiplimab) Compared to Pembrolizumab in the Adjuvant Setting in Patients with Completely Resected High-Risk Melanoma

This present phase 3 study assesses the combination of adjuvant fianlimab and cemiplimab versus pembrolizumab in patients with completely resected high-risk melanoma.

### Objectives

The primary objective of the study is to demonstrate superiority of fianlimab+cemiplimab compared to pembrolizumab, as measured by relapse-free survival (RFS).

Secondary objectives include the following:
To demonstrate superiority of fianlimab+cemiplimab compared to pembrolizumab, as measured by overall survival (OS).
To demonstrate superiority of fianlimab+cemiplimab compared to pembrolizumab, as measured by melanoma-specific survival (MSS).
To evaluate whether post-operative adjuvant therapy improves distant metastasis-free survival (DMFS), in stage IIC or III patients receiving fianlimab+cemiplimab compared to pembrolizumab.
To assess impact of fianlimab+cemiplimab on quality of life as compared to pembrolizumab in adults.
To assess safety and tolerability of fianlimab+cemiplimab compared to pembrolizumab.
To characterize pharmacokinetics (PK) of fianlimab+cemiplimab using sparse PK sampling in patients 12 years of age and older.
To assess immunogenicity of fianlimab and against cemiplimab.

### Study Design

This randomized, double-blind, phase 3 study in patients 12 years of age or older with completely resected high-risk melanoma in the adjuvant setting has three arms:
Arm A: fianlimab (1600 mg)+cemiplimab (350 mg) every 3 weeks (Q3W) IV
Arm B: fianlimab (400 mg)+cemiplimab (350 mg) Q3W IV
Arm C: pembrolizumab (200 mg)+saline/dextrose placebo Q3W IV

Patients are randomized 1:1:1 to Arm A, Arm B, and Arm C. Patients have had no prior systemic therapy for melanoma and have fully resected melanoma of stages IIC, III or IV (American Joint Committee on Cancer [AJCC] v8).

The study population is patients within 12 weeks of full surgical resection of high-risk stage IIC, III, or IV melanoma (8th Edition of American Joint Committee on Cancer melanoma classification, 2017) (Amin, 2017).

### Inclusion Criteria

A patient must meet the following criteria to be eligible for inclusion in the study:

1. At least 12 years of age on the date of providing informed consent. Note: Patients who are <18 years will be included in the territories where accepted per local laws, regulations and ethics committee.

2. All patients must be either stage IIC, III, or stage IV per AJCC 8th edition (Amin, 2017) and have histologically confirmed melanoma that is completely surgically resected in order to be eligible.
Patients with stage IIIA disease must have at least 1 lymph node micrometastasis measuring >1 mm in greatest diameter.
Patients with stage IIC, IIIA, and IV (M1c/d) are each capped at 10% of the total population (i.e., 30% in total).
Patients with acral and mucosal melanoma are allowed, provided that they underwent complete resection, with 10% cap total for both types.
Patients with melanoma of unknown primary may be allowed, provided that they underwent complete resection.
Stage IIC melanoma must be confirmed by a pathologically negative sentinel lymph node biopsy (SLNB) specimen and no evidence of regional or distant metastasis.

3. Complete surgical resection must be performed within 12 weeks prior to treatment, and enrollment may occur only after satisfactory wound healing from the surgery.
Complete surgical resection is defined as negative microscopic margins on resected primary tumors along with SLNB. Complete lymph node dissection in the case of a positive SLNB is not mandatory provided that patient is clinically lymph node negative and resection margins are microscopically clear. Surgical/pathology reports documenting complete resection must be reviewed and signed by the Investigator before study entry for all patients.
A solitary skin melanoma lesion without epithelial component should be treated as a primary tumor and must undergo SLNB.
For all resected stage IV Mid patients, neurosurgical and pathology reports documenting complete resection and clear margins must be reviewed and signed by the Investigator prior to enrollment.

4. All patients must have disease-free status documented by a complete physical examination and imaging studies within 4 weeks prior to treatment. Imaging must include a scan of the chest, abdomen, pelvis (preferably using contrast X-ray CT), and of all relevant anatomical areas and sites of resected disease (for patients with stage II and IV disease). Contrast-enhanced MRI of brain must be performed at staging for all patients.
For patients with abnormal or suspicious findings in the screening scans, residual malignancy and/or metastatic disease must be excluded by ¹⁸F-FDG PET-CT and/or biopsy.
For patients with resected stage IV M1d disease, repeat brain MRI with contrast showing no evidence of residual disease or disease relapse must be performed at least 4 weeks post-surgery/post-RT, and prior to study entry.

5. Patients must not have received systemic anti-cancer therapy or radiation therapy for melanoma in the previous 5 years, with the exception of stage IV M1d patients who may have had RT to the CNS following radical resection. Adjuvant radiation therapy to the primary site and/or to the nodal bed following radical surgical resection is allowed where it is considered institutional standard of care.

6. Performance status:
For adult patients: ECOG PS 0 or 1.
For patients <18 years of age: Karnofsky PS>70 (patients >16 years) or Lansky PS>70 (patients <16 years).

7. Adequate bone marrow function, as determined by hematological parameters:
Absolute neutrophil count (ANC)≥1.5×10\hat{ }9/L (1500/mm³)
Hemoglobin ≥9.0 g/dL (5.59 mmol/L)
Platelet count 75,000/mm³

8. Adequate hepatic function, as determined by:
AST/ALT for adults: aspartate aminotransferase (AST)≤3× upper limit of normal (ULN) and alanine aminotransferase (ALT)≤3×ULN
AST/ALT for adolescents: AST and ALT<2.5 ULN
serum bilirubin≥1.5×ULN, except in patients with clinically documented Gilbert's Syndrome where ≤3× the ULN is permitted

9. Adequate kidney function:
For adult patients: as determined by an estimated glomerular filtration rate (eGFR)≥30 ml/min (using the creatine phosphokinase (CPK)-EPI equation)
For pediatric patients:
   (i) Creatinine clearance or radioisotope glomerular filtration rate (GFR)>70 mL/min/1.73 m²
or
   (ii) Serum creatinine based on age/gender as shown in Table 1:

TABLE 1
Serum Creatinine Based on Age/Gender

Maximum Serum
Creatinine (mg/dL)

| Age | Male | Female |
|---|---|---|
| 12 to <13 | 1.2 | 1.2 |
| years | | |
| 13 to <16 | 1.5 | 1.4 |
| years | | |
| ≥16 years | 1.7 | 1.4 |

Threshold creatinine values derived from the Schwartz formula for GFR estimation, which utilizes child body length/stature data from the Centers for Disease Control (Schwartz, 1985)

10. All patients must provide a minimum of 25 slides or 125 mm³ of a resected tumor tissue sample (FFPE) from tissue block for biomarker analyses. Cut slides must be shipped to testing lab within 2 weeks of slide preparation. Bone biopsies are not allowed.

11. Women of childbearing potential (WOCBP*) must have a negative serum (beta-human chorionic gonadotropin [beta-hCG]) at screening, and must agree to not get pregnant during the study treatment and for up to 6 months after receiving last dose of study treatment.

*WOCBP are defined as women who are fertile following menarche until becoming postmenopausal, unless permanently sterile. Permanent sterilization methods include hysterectomy, bilateral salpingectomy, and bilateral oophorectomy.

A postmenopausal state is defined as no menses for 12 months without an alternative medical cause. A high follicle stimulating hormone (FSH) level in the postmenopausal range may be used to confirm a postmenopausal state in women not using hormonal contraception or hormonal replacement therapy. However, in the absence of 12 months of amenorrhea, a single FSH measurement is insufficient to determine the occurrence of a postmenopausal state. The above definitions are according to the Clinical Trial Facilitation Group (CTFG) guidance. Pregnancy testing and contraception are not required for women with documented hysterectomy.

Male study participants with WOCBP partners are required to use condoms for 6 months after receiving the last therapy dose unless they are vasectomized or practice sexual abstinence.

Vasectomized partner or vasectomized study participant must have received medical assessment of the surgical success.

Periodic abstinence (calendar, symptothermal, post-ovulation methods), withdrawal (coitus interruptus), spermicides only, and lactational amenorrhea method (LAM) are not acceptable methods of contraception. Female condom and male condom should not be used together.

12. WOCBP must agree not to donate eggs (ova, oocytes) for the purposes of assisted reproduction during the entire trial and until 6 months after last treatment.

13. All men must agree not to donate sperm during the trial and for 6 months after receiving the last therapy dose.

14. Provide informed consent signed by study patient (patients aged 12-17 provide assent complemented by parent or legal guardian consent).

15. Willing and able to comply with clinic visits and study-related procedures.

16. Able to understand and complete study-related questionnaires.

### Exclusion Criteria

A patient who meets any of the following criteria is excluded from the study:

1. Uveal melanoma.

2. Any evidence of residual disease after surgery by imaging, pathology, or cytology (including clinical or radiological suspicion of leptomeningeal metastasis).

3. Ongoing or recent (within 2 years) evidence of clinically significant autoimmune disease that required systemic treatment with immunosuppressive agents. The following are non-exclusionary: vitiligo, childhood asthma that has resolved, residual hypothyroidism that requires only hormone replacement, psoriasis not requiring systemic treatment.

4. Uncontrolled infection with HIV, HBV, or HCV infection; or diagnosis of immunodeficiency that is related to, or results in chronic infection.

### Notes:

Patients with known HIV who have controlled infection (undetectable viral load and CD4 count above 350 either spontaneously or on a stable antiviral regimen) are permitted. For patients with controlled HIV infection, monitoring is performed per local standards.
Patients with known hepatitis B (HepBsAg+) who have controlled infection (serum hepatitis B virus DNA PCR that is below the limit of detection AND receiving anti-viral therapy for hepatitis B) are permitted. Patients with controlled infections must undergo periodic monitoring of HBV DNA per local standards and must remain on anti-viral therapy for at least 6 months beyond the last dose of investigational study drug.
Patients who are known HCV Ab+ who have controlled infection (undetectable HCV RNA by PCR either spontaneously or in response to a successful prior course of anti-HCV therapy) are permitted.
Patients with HIV or hepatitis must be reviewed by a qualified specialist (e.g., infectious disease or hepatologist) managing this disease prior to commencing and regularly throughout the duration of their participation in the trial.

5. Another malignancy that is currently progressing or that required active treatment in the past 5 years, with the exception of those with a negligible risk of metastasis or death (such as adequately treated carcinoma in situ of the cervix, basal or squamous cell skin cancer, localized early-stage prostate cancer, or ductal carcinoma in situ of the breast). Note: any uncertain case should be discussed with the Medical Monitor before enrollment.

6. Pregnant or breastfeeding women.

7. WOCBP who are unwilling to practice highly effective contraception prior to the initial dose/start of the first treatment, during the study, and for at least 6 months after the last dose. Highly effective contraceptive measures include:
Stable use of combined (estrogen and progestogen containing) hormonal contraception (oral, intravaginal, transdermal) or progestogen-only hormonal contraception (oral, injectable, implantable) associated with inhibition of ovulation initiated 2 or more menstrual cycles prior to screening;
intrauterine device (IUD); intrauterine hormone-releasing system (IUS);
bilateral tubal ligation (occlusion);
vasectomized partner (provided that the male vasectomized partner is the sole sexual partner of the WOCBP study participant and that the vasectomized partner has obtained medical assessment of surgical success for the procedure); and/or
sexual abstinence †, ‡.
   (i) †Sexual abstinence is considered a highly effective method only if defined as refraining from heterosexual intercourse during the entire period of risk associated with the study drugs. The reliability of sexual abstinence needs to be evaluated in relation to the duration of the clinical trial and the preferred and usual lifestyle of the subject.
   (ii) ‡Periodic abstinence (calendar, symptothermal, post-ovulation methods), withdrawal (coitus interruptus), spermicides only, and lactational amenorrhea method (LAM) are not acceptable methods of contraception. Female condom and male condom should not be used together.

### Prior/Concomitant Therapy:

8. Systemic immune suppression:
Use of immunosuppressive doses of corticosteroids (>10 mg of prednisone per day or equivalent) within 14 days of the first dose of study medication. Physiologic replacement doses are allowed up to and including 10 mg of prednisone/day or equivalent. Inhaled or topical steroids are permitted.
Other clinically relevant forms of systemic immune suppression.

9. Treatment with any anti-cancer therapy for malignancies other than melanoma, including immuno-therapy, chemotherapy, radiotherapy, or biological therapy in the 5 years prior to treatment. Adjuvant hormonotherapy used for breast cancer or other hormone-sensitive cancers in long-term remission is allowed.

### Other Comorbidities:

10. History or current evidence of significant (CTCAE Grade ≥2) local or systemic infection (e.g, cellulitis, pneumonia, septicemia) requiring systemic antibiotic treatment within 2 weeks prior to the first dose of trial medication.

### Other Exclusions:

11. Known hypersensitivity to the active substances or to any of the excipients, or any contraindications to either cemiplimab or pembrolizumab per local prescribing information.

12. Presence of a severe concurrent illness or other condition (eg, psychological, family, sociological, or geographical circumstances) that does not permit adequate follow-up and compliance with the protocol.

13. Cardiovascular disease, as defined below:
New York Heart Association (NYHA) heart failure classifications of Class II, III, or IV; or
Myocardial infarction (MI) or acute coronary syndrome (ACS) within 6 months; or
Transient ischemic attack (TIA) or stroke within 1 year.

14. Received a live vaccine within 30 days of planned start of study medication. Note: Live or live attenuated vaccination with replicating potential. If a patient intends to receive a COVID-19 vaccine before the start of study drug, participation in the study should be delayed at least 1 week after any COVID-19 vaccination. During the treatment period, it is recommended to delay COVID-19 vaccination until patients are receiving and tolerating the study drug. A booster vaccine dose should not be less than 48 hours before or after study drug dosing.

15. Major surgical procedure (i.e., requiring general anesthesia), or significant traumatic injury within 4 weeks prior to screening.

16. Exception: Melanoma-related minor surgery/biopsy are allowed, provided that there is satisfactory wound healing before receiving study treatment.

17. Prior allogeneic stem cell transplant or solid organ transplant

18. Any medical condition that in the opinion of the Investigator would make participation in the study not in the best interest of the patient.

19. Members of the clinical site study team and/or his/her immediate family, unless prior approval granted by the Sponsor.

20. Patients who are committed to an institution by virtue of an order issued either by the judicial or the administrative authorities are excluded from this study.

21. Adolescent patients (≥12 to <18 years old) with body weight <40 kg.

### Dose/Route/Schedule:

The combination of cemiplimab plus fianlimab and the combination of pembrolizumab plus placebo are prepared by an unblinded pharmacist at the investigative site and administered in a blinded fashion, in an outpatient setting. Adult and adolescent patients receive study drug co-infusion in a 30 minute (±10 minute) IV infusion Q3W. Similarly, pembrolizumab and placebo are infused in a 30 minute (±10 minute) IV infusion once every three weeks.

### Fianlimab and Cemiplimab (for Combination Co-Infusion)

Fianlimab 1600 mg is supplied as a liquid in sterile, single-use vial. Instructions on dose preparation are provided in the pharmacy manual.

Fianlimab 400 mg is supplied as a liquid in sterile, single-use vial. Instructions on dose preparation are provided in the pharmacy manual.

Cemiplimab 350 mg is supplied as a liquid in sterile, single-use vial. Instructions on dose preparation are provided in the pharmacy manual.

### Pembrolizumab

Pembrolizumab 200 mg is prepared for co-infusion (with saline/dextrose placebo) at the investigative sites as a liquid in a sterile, single-use vial. Adolescent patients receive pembrolizumab at 2 mg/kg (to maximum 200 mg total). Instructions on dose preparation are provided in the pharmacy manual.

### Endpoints

The primary endpoint for all patients is investigator-assessed RFS, defined as the time from treatment to the first documented recurrence of disease at any site (excluding new primary melanomas) or death from any cause, whichever occurs first.

For efficacy, the key secondary endpoint is overall survival, defined as the time from treatment to the date of death. Additional efficacy endpoints include melanoma specific survival, defined as death due to melanoma (deaths due to other causes or unclear causes are censored), and distant metastasis free survival, defined as the time between the date of treatment and the date of the first distant metastasis.

### Patient Reported Outcomes

The global health status secondary endpoints include patient-reported outcomes (PROs) for adult patients, as measured by European Organization for Research and Treatment of Cancer Quality of Life Questionnaire C30 (EORTC-QLQ-C30), European Quality of Life Dimension 5 (EQ-5D-5L), Functional Assessment of Cancer Therapy (FACT)—melanoma (melanoma subscale only), Patient Global Impressions Scale (PGIS), and Patient Global Impressions of Change Scale (PGIC).

### Procedures and Assessments

The primary endpoint is RFS, which is an accepted primary endpoint in this clinical setting. The secondary efficacy endpoints include OS, MSS, DMFS, and PROs, in adults only, as measured by EORTC QLQ-C30, EQ-5D-5L, FACT-melanoma (melanoma subscale only), the PRO CTCAE for fatigue, PGIS, and PGIC). The Pediatric Quality of Life Inventory (PEDSQL) is administered in adolescents. Analysis of RFS is based on investigator assessment using imaging or pathology assessment at the time of disease recurrence or upon patient death.

Potential disease relapse that is first detected clinically is assessed by CT or MRI. For solitary suspected lesions or for lesions of unclear etiology and detected by imaging, biopsies are required for histologic confirmation of relapse, if medically appropriate. For cutaneous lesions, biopsy for histologic confirmation of recurrence of disease is also required, if medically appropriate. New cases of primary melanoma are distinguished from recurrences by local pathologists by reviewing skin lesion biopsy samples and resection samples to identify existence of an intraepidermal component and whether they are consistent with a new primary melanoma as opposed to a regional or distant metastatic recurrence following established dermatology practice guidelines.

Blood samples to measure fianlimab and cemiplimab in serum and immunogenicity (presence of ADA and NAb to fianlimab and cemiplimab in serum) are collected from all patients.

Blood samples are collected for analysis of additional biomarkers in plasma and serum. Potential pharmacodynamic, predictive and prognostic biomarkers related to fianlimab and cemiplimab treatment exposure, clinical activity, and/or underlying disease are investigated in plasma, serum, and tumor tissue.

The safety and tolerability of fianlimab in combination with cemiplimab are monitored by clinical assessment of treatment-emergent adverse events (TEAEs)/immune mediated adverse events (imAEs), serious adverse event (SAEs), adverse event of special interest (AESIs), and by repeated measurements and clinical evaluation of vital signs (temperature, blood pressure, pulse, and respiration), physical examinations, 12-lead electrocardiograms (ECGs), and laboratory assessments including standard hematology, chemistry, urinalysis, and other lab tests (including blood cortisol and thyroid-stimulating hormone [TSH]).

### Results

Surprisingly, the combination of fianlimab (REGN3767, anti-LAG-3) and cemiplimab Q3W improves relapse-free survival (RFS) compared to pembrolizumab in the adjuvant setting in patients with completely resected high-risk melanoma.

### Example 2: Phase 3 Clinical Trial of Anti-LAG-3 Antibody (REGN3767, Fianlimab) and Anti-PD-1 Antibody (REGN2810, Cemiplimab) in Patients with Previously Untreated Unresectable Locally Advanced or Metastatic Melanoma

This present phase 3 study assesses the combination of fianlimab and cemiplimab versus pembrolizumab in patients with previously untreated unresectable locally advanced or metastatic melanoma.

### Objectives

The primary objective of the present study is to demonstrate superiority of fianlimab 1600 mg+cemiplimab and/or fianlimab 400 mg+cemiplimab compared to pembrolizumab, as measured by progression-free survival (PFS).

Secondary objectives include the following:
To demonstrate superiority of fianlimab 1600 mg+cemiplimab and/or fianlimab 400 mg+cemiplimab compared to pembrolizumab, as measured by overall survival (OS).
To demonstrate superiority in ORR of fianlimab 1600 mg+cemiplimab and/or fianlimab 400 mg+cemiplimab compared to pembrolizumab.
To characterize the ORR, PFS, and OS with fianlimab 1600 mg+cemiplimab and/or fianlimab 400 mg+cemiplimab compared to cemiplimab to inform the contribution of each components.
To assess immunogenicity of fianlimab 1600 mg and/or 400 mg and cemiplimab.
To assess impact of fianlimab 1600 mg+cemiplimab and/or fianlimab 400 mg+cemiplimab on physical functioning and role functioning and global health status/quality of life, as compared to pembrolizumab in adults.
To characterize safety and tolerability of treatment in patients 12 to <18 years of age.
To characterize ORR, PFS, and OS with treatment in patients 12 to <18 years of age.
To assess the safety and tolerability of fianlimab+cemiplimab compared to pembrolizumab and to cemiplimab.
To characterize pharmacokinetics (PK) of treatment using sparse PK sampling in patients aged ≥12 years.

### Study Design

This is a phase 3 study in patients aged ≥12 years with unresectable locally advanced or metastatic melanoma who have not received a previous systemic treatment for advanced disease.

For adult and adolescent patients there are 4 main arms to the study, which is conducted in a randomized, double-blind fashion:
Arm A: fianlimab (1600 mg every 3 weeks [Q3W], intravenously, [IV])+cemiplimab (350 mg Q3W IV)
Arm A1: fianlimab (400 mg every 3 weeks [Q3W], intravenously, [IV])+cemiplimab (350 mg Q3W IV)
Arm B: pembrolizumab (200 mg Q3W IV)+saline/dextrose placebo (placebo)
Arm C: cemiplimab (350 mg Q3W IV)+saline/dextrose placebo (placebo)

The study population includes patients with unresectable Stage III or IV (metastatic) melanoma who have not received prior systemic anti-cancer therapy for advanced unresectable and metastatic disease (8th Edition of American Joint Committee on Cancer melanoma classification). The patient population includes male and female patients aged 12 years.

### Inclusion Criteria

A patient must meet the following criteria to be eligible for inclusion in the study:

1. Age ≥12 years on the date of providing informed consent. Note: Patients who are <18 years are included in the territories where accepted per local laws, regulations and Ethics Committees.

2. Patients with histologically confirmed unresectable Stage Ill and Stage IV (metastatic) melanoma per AJCC, eighth revised edition (Amin, 2017) who have not received prior systemic therapy for advanced unresectable disease.
Patients who received adjuvant and/or neoadjuvant systemic therapies are eligible if they did not have evidence of progression or recurrence of disease and/or discontinued due to occurrence of immune related adverse events (irAEs) ≥grade 3 (with the exclusion of endocrinopathies which are fully controlled by hormone replacement) while on such therapies. Also, patients must have had a treatment-free and disease-free interval of >6 months.
Patients with acral and mucosal melanomas are eligible. Accrual is limited to approximately 10% of the total population.

3. Measurable disease per RECIST v1.1
Previously irradiated lesions can only be counted as target lesions if they have been demonstrated to progress and no other target lesion is available
Cutaneous lesions should be evaluated as non-target lesions.

4. Performance status:
For adult patients: Eastern Cooperative Oncology Group (ECOG) performance status (PS) 0 or 1
For pediatric patients: Karnofsky performance status ≥70 (patients ≥16 years) or Lansky performance status ≥70 (patients <16 years)

5. Anticipated life expectancy of at least 3 months.

6. Adequate bone marrow function, as determined by hematological parameters:
Absolute neutrophil count (ANC)≥21.5×10\hat{ }9/L (1500/mm³)
Hemoglobin ≥9.0 g/dL (5.59 mmol/L).
Platelet count ≥75,000/mm³.

7. Adequate hepatic function, as determined by:
AST/ALT for adults: aspartate aminotransferase (AST)≤3×ULN, alanine aminotransferase (ALT)≤3×ULN and alkaline phosphatase <2.5×ULN (or <5×ULN, if liver or bone metastases are present)
AST/ALT for adolescents: AST, ALT and ALP≤2.5 ULN (and without hepatic involvement of tumor) (or <5×ULN, if liver metastases or bone metastases are present)
serum bilirubin ≤1.5×ULN, except in patients with clinically documented Gilbert's Syndrome where ≤3× the ULN is permitted

8. Adequate kidney function:
For adult patients: as determined by an estimated glomerular filtration rate (eGFR) ≥30 mL/min (using the CPK-EPI equation)
For pediatric patients:
   (i) Creatinine clearance or radioisotope glomerular filtration rate (GFR)≥70 mL/min/1.73 m²
or
   (ii) Serum creatinine based on age/gender as shown in Table 2:

TABLE 2
Serum Creatinine Based on Age and Gender

Maximum Serum
Creatinine (mg/dL)

| Age | Male | Female |
|---|---|---|
| 12 to <13 | 1.2 | 1.2 |
| years | | |
| 13 to <16 | 1.5 | 1.4 |
| years | | |
| ≥16 years | 1.7 | 1.4 |

Threshold creatinine values derived from the Schwartz formula for GFR estimation, which utilizes child body length/stature data from the Centers for Disease Control (Schwartz G J, 1985)

9. Biopsy LAG-3 IHC result requirements: A patient must have a valid, centrally-tested, LAG-3 IHC result, to be enrolled into the study. Any LAG-3 level (0-100% expression) is allowed.

### Sample Requirements:

Patients must submit, before enrollment, a tumor tissue sample (formalin fixed paraffin embedded [FFPE]) from tissue block not older than 1 year with no interim therapy. Cut slides must be shipped to testing lab within 2 weeks of slide preparation.

If archival tissue is unavailable or insufficient for testing, a new biopsy may be requested before enrollment, as long as it is deemed by the site investigator that it does not present a potential for serious risk to the health, safety, or welfare of a subject. If unable to obtain archival or new biopsy tissue, the patient is not eligible for enrollment.

The lesion amenable to biopsy has to be a non-target lesion, must have either never received previous radiotherapy or have histological evidence of viable tumor after the last course of radiotherapy. Bone biopsies are not allowed.

10. Women of childbearing potential (WOCBP*) must have a negative serum (beta-human chorionic gonadotropin [beta-hCG]) at screening.

*WOCBP are defined as women who are fertile following menarche until becoming postmenopausal, unless permanently sterile. Permanent sterilization methods include hysterectomy, bilateral salpingectomy, and bilateral oophorectomy.

A postmenopausal state is defined as no menses for 12 months without an alternative medical cause. A high follicle stimulating hormone (FSH) level in the postmenopausal range may be used to confirm a postmenopausal state in women not using hormonal contraception or hormonal replacement therapy. However, in the absence of 12 months of amenorrhea, a single FSH measurement is insufficient to determine the occurrence of a postmenopausal state. The above definitions are according to the Clinical Trial Facilitation Group (CTFG) guidance. Pregnancy testing and contraception are not required for women with documented hysterectomy.

Male study participants with WOCBP partners are required to use condoms for 6 months unless they are vasectomized or practice sexual abstinence.
Vasectomized partner or vasectomized study participant must have received medical assessment of the surgical success.
Periodic abstinence (calendar, symptothermal, post-ovulation methods), withdrawal (coitus interruptus), spermicides only, and lactational amenorrhoea method (LAM) are not acceptable methods of contraception. Female condom and male condom should not be used together.

11. WOCBP must agree not to donate eggs (ova, oocytes) for the purposes of assisted reproduction during the entire trial and until 6 months after last treatment

12. All men must agree not to donate sperm during the trial and for 6 months after receiving the last therapy dose

13. Provide informed consent signed by study patient (patients aged 12-17 provide assent complemented by parent or legal guardian consent if they are NOT incapacitated due to a mental disability).

14. Willing and able to comply with clinic visits and study-related procedures.

15. Able to understand and complete study-related questionnaires.

### Exclusion Criteria

A patient who meets any of the following criteria is excluded from the study:

### Medical Conditions

1. Uveal melanoma

2. Ongoing or recent (within 2 years) evidence of an autoimmune disease that required systemic treatment with immunosuppressive agents. The following are non-exclusionary: vitiligo, childhood asthma that has resolved, residual hypothyroidism the requires only hormone replacement, psoriasis not requiring systemic treatment.

3. Uncontrolled infection with human immunodeficiency virus (HIV), hepatitis B (HBV) or hepatitis C virus (HCV) infection; or diagnosis of immunodeficiency that is related to, or results in chronic infection.

### Notes:

Patients with known HIV who have controlled infection (undetectable viral load and CD4 count above 350 either spontaneously or on a stable antiviral regimen) are permitted. For patients with controlled HIV infection, monitoring is performed per local standards
Patients with known hepatitis B (HepBsAg+) who have controlled infection (serum hepatitis B virus DNA PCR that is below the limit of detection AND receiving anti-viral therapy for hepatitis B) are permitted. Patients with controlled infections must undergo periodic monitoring of HBV DNA per local standards and must remain on anti-viral therapy for at least 6 months beyond the last dose of investigational study drug
Patients who are known hepatitis C virus antibody positive (HCV Ab+) who have controlled infection (undetectable HCV RNA by PCR either spontaneously or in response to a successful prior course of anti-HCV therapy) are permitted.
Patients with HIV or hepatitis must be reviewed by a qualified specialist (e.g., infectious disease or hepatologist) managing this disease prior to commencing and regularly throughout the duration of their participation in the trial

4. Unknown BRAF V600 mutation status. Patients with BRAF-mutated melanoma who present with symptoms of rapidly progressive disease and are considered by Investigator's assessment as likely to benefit from upfront treatment with BRAF/MEK-inhibitors should not be enrolled in the study.

5. Another malignancy that is progressing or required active treatment in the past 2 years, with the exception of those with a negligible risk of metastasis or death (such as adequately treated carcinoma in situ of the cervix, basal or squamous cell skin cancer, localized prostate cancer, or ductal carcinoma in situ).

Note: should be discussed with Medical Monitor in case of uncertainty.

6. Pregnant or breastfeeding women.

7. WOCBP who are unwilling to practice highly effective contraception prior to the initial dose/start of the first treatment, during the study, and for at least 6 months after the last dose. Highly effective contraceptive measures include:
stable use of combined (estrogen and progestogen containing) hormonal contraception (oral, intravaginal, transdermal) or progestogen-only hormonal contraception (oral, injectable, implantable) associated with inhibition of ovulation initiated 2 or more menstrual cycles prior to screening;
intrauterine device (IUD); intrauterine hormone-releasing system (IUS);
bilateral tubal ligation;
vasectomized partner (provided that the male vasectomized partner is the sole sexual partner of the WOCBP study participant and that the vasectomized partner has obtained medical assessment of surgical success for the procedure); and/or
sexual abstinence †, ‡.
   (i) †Sexual abstinence is considered a highly effective method only if defined as refraining from heterosexual intercourse during the entire period of risk associated with the study drugs. The reliability of sexual abstinence needs to be evaluated in relation to the duration of the clinical trial and the preferred and usual lifestyle of the patient.
   (ii) ‡Periodic abstinence (calendar, symptothermal, post-ovulation methods), withdrawal (coitus interruptus), spermicides only, and lactational amenorrhea method (LAM) are not acceptable methods of contraception. Female condom and male condom should not be used together.

### Prior/Concomitant Therapy

8. Systemic immune suppression:
Use of immunosuppressive doses of corticosteroids (≤10 mg of prednisone per day or equivalent) within 14 days of the first dose of study medication. Physiologic replacement doses are allowed up to and including 10 mg of prednisone/day or equivalent. Inhaled or topical steroids are permitted, if they are not for treatment of an autoimmune disorder.
Other clinically relevant forms of systemic immune suppression.

9. Treatment with other anti-cancer therapy including immuno-therapy, chemotherapy, major surgery or biological therapy within 21 days prior to the first dose of trial treatment. Adjuvant hormonotherapy used for breast cancer or other hormone-sensitive cancers in long term remission is allowed.

10. Patients with prior history of Grade 3 immune-mediated adverse events (with the exclusion of endocrinopathies which are fully controlled by hormone replacement) from prior checkpoint inhibitor therapies are excluded.

### Other Comorbidities

11. History or current evidence of significant (CTCAE grade ≥2) local or systemic infection (e.g., cellulitis, pneumonia, septicemia) requiring systemic antibiotic treatment within 14 days prior to the first dose of trial medication.

12. Active or untreated brain metastases or spinal cord compression. Patients with leptomeningeal disease are excluded. Patients with known brain metastases are eligible if they:
received radiotherapy or another appropriate standard therapy for the brain metastases,
have neurologically returned to baseline (except for residual signs and symptoms related to the CNS treatment) for at least 14 days prior to enrollment.
did not require immunosuppressive doses of corticosteroids therapy (>10 mg of prednisone per day or equivalent) in the 14 days prior to enrollment.

Note: Patients who are asymptomatic with a single untreated brain metastasis <10 mm in size are eligible.

### Other Exclusions

13. Known hypersensitivity to the active substances or to any of the excipients

14. Presence of a severe concurrent illness or other condition (e.g., psychological, family, sociological, or geographical circumstances) that does not permit adequate follow-up and compliance with the protocol.

15. Received a live vaccine within 30 days of planned start of study medication, i.e., live or live attenuated vaccination with replicating potential. If a patient intends to receive a COVID-19 vaccine before the start of study drug, participation in the study should be delayed at least 1 week after any COVID-19 vaccination. During the treatment period, it is recommended to delay COVID-19 vaccination until patients are receiving and tolerating a steady dose of study drug. A vaccine dose should not be less than 48 hours before or after study drug dosing.

16. Major surgical procedure, open biopsy, or significant traumatic injury within 4 weeks prior to screening.

17. Prior allogeneic stem cell transplant or solid organ transplant.

18. Any medical condition that in the opinion of the investigator would make participation in the study not in the best interest of the patient.

19. Members of the clinical site study team and/or his/her immediate family, unless prior approval granted by the Sponsor.

20. Patients who are committed to an institution by virtue of an order issued either by the judicial or the administrative authorities are excluded from this study.

21. Adolescent patients (≥12 to <18 years old) with body weight <40 kg.

### Dose/Route/Schedule

The combination of cemiplimab plus fianlimab, the combination of pembrolizumab plus placebo and the combination of cemiplimab plus placebo are prepared by an unblinded pharmacist at the investigative site and are administered in a blinded fashion for all patients.

All infusions for adults and adolescents are administered as a 30 minute (±10 minutes) IV infusion in an outpatient setting, every 3 weeks.

### Cemiplimab

Cemiplimab 350 mg once every three weeks IV is the approved regimen (LIBTAYO®, cemiplimab) for treatment of cutaneous squamous cell carcinoma (CSCC), basal cell carcinoma (BCC), and non-small cell lung carcinoma (NSCLC).

Cemiplimab is supplied as a liquid in sterile, single-use vials. Instructions on dose preparation are provided in the pharmacy manual.

### Fianlimab and Cemiplimab (for Combination/Co-Infusion Administration)

Based on efficacy and safety data from an ongoing phase 1 study, the fianlimab 1600 mg Q3W IV dose with cemiplimab 350 mg Q3W IV was recommended to advance to phase 3. The efficacy of fianlimab 400 mg Q3W IV with cemiplimab 350 mg Q3W IV has not been studied but is being added as it is in line with the expectation to consider lower doses in oncology setting and also consistent with the doses used for other anti-LAG-3 antibodies that are being utilized in phase 2 and 3 clinical trials in melanoma and in other solid tumors.

Fianlimab 1600 mg and 400 mg Q3W IV are supplied as a liquid in sterile, single-use vials. Instructions on dose preparation are provided in the pharmacy manual.

Fianlimab and cemiplimab are administered simultaneously as a mixed co-infusion.

### Pembrolizumab

Pembrolizumab 200 mg Q3W IV is an approved dose for the treatment of advanced and metastatic melanoma.

For adolescent patients randomized to Arm B, pembrolizumab is dosed based on body weight at 2.0 mg/kg Q3W IV (max. 200 mg).

Pembrolizumab is prepared for co-infusion (with saline placebo) at the investigative sites as a liquid in sterile. Instructions on dose preparation are provided in the pharmacy manual.

### Endpoints

The primary endpoint is PFS (progression-free survival) (per Response Evaluation Criteria in Solid Tumors (RECIST) 1.1 based on blinded independent central review [BICR]).

The key endpoint for efficacy is overall survival and objective response rate (ORR), defined as the proportion of patients who achieve a best overall response of CR (complete response) or PR (partial response) (per RECIST 1.1, based on BICR). Additional efficacy endpoints include DCR (disease control rate), defined as the proportion of patients who achieve a best overall response of CR or PR or stable disease (SD) (per RECIST 1.1 based on BICR; SD assessed at least 6 months after first dose).

DoR (duration of response), defined as the time from initial response (CR or PR per RECIST 1.1) to first occurrence of PD (progressive disease) (per RECIST 1.1 via BICR), or death due to any cause, whichever occurs first.

PFS, ORR, DCR, and DoR based on investigator assessment according to RECIST 1.1 and iRECIST (immune RECIST).

### Patient Reported Outcomes:

The global health status secondary endpoints are:
Patient-reported outcomes, as measured by EORTC QLQ-C30, EQ-5D-5L, FACT-melanoma (melanoma subscale only), PGIS, and PGIC.
Change from baseline in Physical functioning at Week 25 per EORTC QLQ-C30
Change from baseline in Role functioning at Week 25 per EORTC QLQ-C30
Change from baseline in GHS/QoL at Week 25 per EORTC QLQ-C30
Change from baseline in Physical functioning during the study per EORTC QLQ-C30
Change from baseline in Role functioning during the study per EORTC QLQ-C30
Change from baseline in GHS/QoL during the study per EORTC QLQ-C30.

### Procedures and Assessments

Efficacy endpoints include anti-tumor activity. For all patients in the study, this is assessed by computed tomography (CT) or magnetic resonance imaging (MRI). For cutaneous lesions, digital photography can be used.

The safety and tolerability of fianlimab in combination with cemiplimab is monitored by clinical assessment of TEAEs/immune related adverse events (irAEs), SAEs, AESIs and by repeated measurements and clinical evaluation of vital signs (temperature, blood pressure, pulse, and respiration), physical examinations, 12-lead electrocardiograms (ECGs), and laboratory assessments including standard hematology, chemistry, urinalysis and other lab tests (including blood cortisol and TSH).

Blood samples are collected from all patients to measure functional fianlimab and functional cemiplimab in serum and to measure immunogenicity (presence of ADA and NAb to fianlimab and cemiplimab in serum).

Centrally determined LAG-3 immunohistochemistry (IHC) results are required for patient eligibility. A patient must have a valid, centrally-tested, LAG-3 IHC result, to be enrolled into the study. Any LAG-3 level (0-100% expression) are allowed. Patient samples are collected for analysis of additional biomarkers. Speculated pharmacodynamic and/or predictive and prognostic biomarkers related to fianlimab and cemiplimab treatment exposure; clinical activity; and/or underlying disease may be investigated. Serum, plasma, peripheral blood mononuclear cells (PBMCs), and tumor tissue are collected.

### Results

Surprisingly, the combination of fianlimab (REGN3767, anti-LAG-3) and cemiplimab improves progression-free survival (PFS) compared to pembrolizumab in patients with previously untreated, unresectable locally advanced or metastatic melanoma. Further, it is expected that the combination of fianlimab and cemiplimab demonstrates a similar PK, safety, and efficacy profile in adolescent patients as those profiles observed for adults in patients with previously untreated, unresectable locally advanced or metastatic melanoma.

### Example 3: Phase 3 Clinical Trial Comparing Anti-LAG-3 (Fianlimab) Plus Anti-PD-1 (Cemiplimab) to Pembrolizumab in Patients with Completely Resected High-Risk Melanoma

Background: Melanoma accounts for the majority of skin cancer-related deaths. Most patients with a newly diagnosed melanoma have resectable disease and are potentially cured by surgery. However, regional nodal and/or distant relapses can occur after curative-intent resection. Postoperative adjuvant therapy with immune checkpoint inhibitors improves relapse-free survival (RFS) and distant metastasis-free survival (DMFS) of patients at high risk of melanoma. Fianlimab (anti-LAG-3) and cemiplimab (anti-PD-1) are both high-affinity, fully human, IgG4 monoclonal antibodies (MAbs) that combined have shown high clinical activity in patients with advanced melanoma in a phase 1 study.

Methods: The present study is a three-way, double-blind, phase 3 trial to compare fianlimab+cemiplimab to pembrolizumab in the adjuvant therapy of high-risk, resected melanoma. The primary objective is relapse-free survival, and the secondary objectives are overall survival, safety, pharmacology, and immunogenicity.

Patient eligibilities: (1) ≥12 years of age; (2) Stage IIc, III or IV (all M-stages) and histologically confirmed melanoma, completely resected ≤12 weeks prior to treatment; (3) no prior systemic anti-cancer therapy or radiation therapy for melanoma in the previous 5 years; (4) no evidence of metastatic disease on staging investigations; and (5) an Eastern Cooperative Oncology Group performance status (PS) of 0 or 1 (for adult pts), Karnofsky PS>70 (pts>16 years) or Lansky PS>70 (pts<16 years).

Study arms (all treatments every 3 weeks intravenously for one year): A. fianlimab (1600 mg)+cemiplimab (350 mg); B. fianlimab (400 mg)+cemiplimab (350 mg); C. pembrolizumab (200 mg)+saline/dextrose placebo. The placebo controlled trial enrolls patients, randomized 1:1:1 to Arms A:B:C, treated for up to 1 year. The trial stratifies by disease stage (stage IIIA vs IIC-IIIB-IIIC vs IIID-IV [M1a/b] vs IV [M1c/d]), and geography (North America vs Europe vs Rest of World).

The primary endpoint is investigator-assessed relapse-free survival. The secondary endpoints include efficacy (overall survival, distant metastasis-free survival, melanoma-specific survival), safety [treatment-emergent adverse events (TEAEs), interruption or discontinuation of drugs due to TEAEs], pharmacokinetic (concentrations of fianlimab and cemiplimab in serum over time), immunogenicity (anti-drug antibodies and neutralizing antibodies in serum against fianlimab or cemiplimab), and patient reported outcomes. The analysis is performed when 242 relapse-free survival events have been observed.

Results: The trial is enrolling. Surprisingly, administration of fianlimab in combination with cemiplimab leads to enhanced tumor regression and improved disease control in patients with completely resected high-risk melanoma.

The present disclosure is not to be limited in scope by the specific embodiments described herein. Indeed, various modifications of the disclosure in addition to those described herein will become apparent to those skilled in the art from the foregoing description and the accompanying figures. Such modifications are intended to fall within the scope of the appended claims.

TABLE 3
Informal Sequence Listing

| SEQ. | | |
|---|---|---|
| ID NO. | Sequence | Description |
| 1. | EVQLLESGGV LVQPGGSLRL SCAASGFTFS | Amino acid |
| | NFGMTWVRQA PGKGLEWVSG ISGGGRDTYF | sequence |
| | ADSVKGRFTI SRDNSKNTLY LQMNSLKGED | |
| | TAVYYCVKWG NIYFDYWGQG TLVTVSS | |
| 2. | DIQMTQSPSS LSASVGDSIT ITCRASLSIN TFLNWYQQKP | Amino acid |
| | GKAPNLLIYA ASSLHGGVPS | sequence |
| | RFSGSGSGTD FTLTIRTLQP EDFATYYCQQ | |
| | SSNTPFTFGP GTVVDFR | |
| 3. | GFTFSNFG | Amino acid |
| | | sequence |
| 4. | ISGGGRDT | Amino acid |
| | | sequence |
| 5. | VKWGNIYFDY | Amino acid |
| | | sequence |
| 6. | LSINTF | Amino acid |
| | | sequence |
| 7. | AAS | Amino acid |
| | | sequence |
| 8. | QQSSNTPFT | Amino acid |
| | | sequence |
| 9. | EVQLLESGGV LVQPGGSLRL SCAASGFTFS | Amino acid |
| | NFGMTWVRQA PGKGLEWVSG ISGGGRDTYF | sequence |
| | ADSVKGRFTI SRDNSKNTLY LQMNSLKGED | |
| | TAVYYCVKWG NIYFDYWGQG TLVTVSSAST | |
| | KGPSVFPLAP CSRSTSESTA ALGCLVKDYF | |
| | PEPVTVSWNS GALTSGVHTF PAVLQSSGLY | |
| | SLSSVVTVPS SSLGTKTYTC NVDHKPSNTK | |
| | VDKRVESKYG PPCPPCPAPE FLGGPSVFLF | |
| | PPKPKDTLMI SRTPEVTCVV VDVSQEDPEV | |
| | QFNWYVDGVE VHNAKTKPRE EQFNSTYRVV | |
| | SVLTVLHQDW LNGKEYKCKV SNKGLPSSIE | |
| | KTISKAKGQP REPQVYTLPP SQEEMTKNQV | |
| | SLTCLVKGFY PSDIAVEWES NGQPENNYKT | |
| | TPPVLDSDGS FFLYSRLTVD KSRWQEGNVF | |
| | SCSVMHEALH NHYTQKSLSL SLGK | |
| 10. | DIQMTQSPSS LSASVGDSIT ITCRASLSIN TELNWYQQKP | Amino acid |
| | GKAPNLLIYA ASSLHGGVPS | sequence |
| | RFSGSGSGTD FTLTIRTLQP EDFATYYCQQ | |
| | SSNTPFTFGP GTVVDFRRTV AAPSVFIFPP | |
| | SDEQLKSGTA SVVCLLNNFY PREAKVQWKV | |
| | DNALQSGNSQ ESVTEQDSKD STYSLSSTLT | |
| | LSKADYEKHK VYACEVTHQG LSSPVTKSFN RGEC | |
| 11. | QVQLVESGGG VVQPGRSLRL SCVASGFTFS | Amino acid |
| | SYGMHWVRQA PGKGLEWVAI IWYDGSNKYY | sequence |
| | ADSVKGRFTI SRDNSKNTQY LQMNSLRAED | |
| | TAVYYCASVA TSGDFDYYGM DVWGQGTTVT | |
| | VSS | |
| 12 | EIVLTQSPAT LSLSPGERTT LSCRASQRIS | Amino acid |
| | TYLAWYQQKP GQAPRLLIYD ASKRATGIPA | sequence |
| | RFSGSGSGTG FTLTISSLEP EDFAVYYCQQ | |
| | RSNWPLTFGG GTKVEIK | |
| 13. | GFTFSSYG | Amino acid |
| | | sequence |
| 14. | IWYDGSNK | Amino acid |
| | | sequence |
| 15. | ASVATSGDFD YYGMDV | Amino acid |
| | | sequence |
| 16. | QRISTY | Amino acid |
| | | sequence |
| 17. | DAS | Amino acid |
| | | sequence |
| 18. | QQRSNWPLT | Amino acid |
| | | sequence |
| 19 | QVQLVESGGG VVQPGRSLRL SCVASGFTFS | Amino acid |
| | SYGMHWVRQA PGKGLEWVAI IWYDGSNKYY | sequence |
| | ADSVKGRFTI SRDNSKNTQY LQMNSLRAED | |
| | TAVYYCASVA TSGDFDYYGM DVWGQGTTVT | |
| | VSSASTKGPS VFPLAPCSRS TSESTAALGC | |
| | LVKDYFPEPV TVSWNSGALT SGVHTFPAVL | |
| | QSSGLYSLSS VVTVPSSSLG TKTYTCNVDH | |
| | KPSNTKVDKR VESKYGPPCP PCPAPPVAGP | |
| | SVFLFPPKPK DTLMISRTPE VTCVVVDVSQ | |
| | EDPEVQFNWY VDGVEVHNAK TKPREEQFNS | |
| | TYRVVSVLTV LHQDWLNGKE YKCKVSNKGL | |
| | PSSIEKTISK AKGQPREPQV YTLPPSQEEM | |
| | TKNQVSLTCL VKGFYPSDIA VEWESNGQPE | |
| | NNYKTTPPVL DSDGSFFLYS RLTVDKSRWQ | |
| | EGNVFSCSVM HEALHNHYTQ KSLSLSLGK | |
| 20. | EIVLTQSPAT LSLSPGERTT LSCRASQRIS | Amino acid |
| | TYLAWYQQKP GQAPRLLIYD ASKRATGIPA | sequence |
| | RFSGSGSGTG FTLTISSLEP EDFAVYYCQQ | |
| | RSNWPLTFGG GTKVEIKRTV AAPSVFIFPP | |
| | SDEQLKSGTA SVVCLLNNFY PREAKVQWKV | |
| | DNALQSGNSQ ESVTEQDSKD STYSLSSTLT | |
| | LSKADYEKHK VYACEVTHQG LSSPVTKSFN RGEC | |

We claim:

1. A method of treating melanoma or inhibiting the growth of a melanoma comprising administering to a subject in need thereof a therapeutically effective amount each of (a) an antibody or antigen-binding fragment thereof that specifically binds programmed death 1 (PD-1); and (b) an antibody or antigen-binding fragment thereof that specifically binds lymphocyte activation gene-3 (LAG-3),
wherein the melanoma is unresectable locally advanced melanoma, unresectable metastatic melanoma, or completely resected high-risk melanoma.

2. The method of claim 1, wherein one dose of the anti-PD-1 antibody or antigen-binding fragment thereof comprises 50 to 1500 mg.

3. The method of claim 1 or 2, wherein one dose of the anti-PD-1 antibody or antigen-binding fragment thereof comprises 350 mg.

4. The method of any one of claims 1 to 3, wherein one dose of the anti-LAG-3 antibody or antigen-binding fragment thereof comprises between 50 to 8000 mg.

5. The method of any one of claims 1 to 4, wherein one dose of the anti-LAG-3 antibody or antigen-binding fragment thereof comprises 400 mg.

6. The method of any one of claims 1 to 4, wherein one dose of the anti-LAG-3 antibody or antigen-binding fragment thereof comprises 1600 mg.

7. The method of any one of claims 1 to 6, wherein the anti-LAG-3 antibody or antigen-binding fragment thereof is administered prior to, concurrent with or after the anti-PD-1 antibody or antigen-binding fragment thereof.

8. The method of claim 7, wherein the anti-LAG-3 antibody or antigen-binding fragment thereof is administered prior to the anti-PD-1 antibody or antigen-binding fragment thereof.

9. The method of claim 7, wherein the anti-LAG-3 antibody or antigen-binding fragment thereof is administered the same day as the anti-PD-1 antibody or antigen-binding fragment thereof.

10. The method of any one of claims 1 to 9, wherein two or more doses of the anti-LAG-3 antibody or antigen-binding fragment thereof are administered in combination with two or more doses of the anti-PD-1 antibody or antigen-binding fragment thereof.

11. The method of claim 10, wherein each dose of the anti-PD-1 antibody or antigen-binding fragment thereof comprises 350 mg.

12. The method of claim 10 or 11, wherein each dose of the anti-LAG-3 antibody or antigen-binding fragment thereof comprises between 50 and 8000 mg.

13. The method of any one of claims 10 to 12, wherein each dose of the anti-LAG-3 antibody or antigen-binding fragment thereof comprises 1600 mg.

14. The method of any one of claims 10 to 12, wherein each dose of the anti-LAG-3 antibody or antigen-binding fragment thereof comprises 400 mg.

15. The method of claim 10, wherein each dose of the anti-PD-1 antibody or antigen-binding fragment thereof comprises 200 mg, 250 mg or 350 mg and each dose of the anti-LAG-3 antibody or antigen-binding fragment thereof comprises 400 mg, 800 mg, 1000 mg, 1400 mg, or 1600 mg.

16. The method of any one of claims 10 to 15, wherein each dose of the anti-PD-1 antibody or antigen-binding fragment thereof is administered 0.5 weeks to 12 weeks after the immediately preceding dose.

17. The method of any one of claims 10 to 16, wherein each dose of the anti-LAG-3 antibody or antigen-binding fragment thereof is administered 0.5 weeks to 12 weeks after the immediately preceding dose.

18. The method of any one of claims 10 to 17, wherein each dose of the anti-PD-1 antibody or antigen-binding fragment thereof is administered once in six weeks.

19. The method of any one of claims 10 to 18, wherein each dose of the anti-LAG-3 antibody or antigen-binding fragment thereof is administered once in six weeks.

20. The method of any one of claims 10 to 17, wherein each dose of the anti-PD-1 antibody or antigen-binding fragment thereof is administered once in three weeks.

21. The method of any one of claims 10 to 18 or 20, wherein each dose of the anti-LAG-3 antibody or antigen-binding fragment thereof is administered once in three weeks.

22. The method of any one of claims 1 to 21, wherein the antibodies are administered intravenously, subcutaneously, or intraperitoneally.

23. The method of any one of claims 1 to 22, wherein the melanoma is unresectable locally advanced melanoma.

24. The method of any one of claims 1 to 22, wherein the melanoma is unresectable metastatic melanoma.

25. The method of claim 23 or 24, wherein the patient is further selected as having one or more of the following criteria:
(i) at least 12 years of age on the date of providing informed consent;
(ii) either stage IIC, III, or stage IV per AJCC 8th edition (Amin, 2017) and have histologically confirmed melanoma that is completely surgically resected;
(iii) patients with stage IIIA disease must have at least 1 lymph node micrometastasis measuring >1 mm in greatest diameter;
(iv) Stage IIC melanoma confirmed by a pathologically negative Sentinel lymph node biopsy (SLNB) specimen and no evidence of regional or distant metastasis;
(v) complete surgical resection performed within 12 weeks prior to treatment, and treatment may occur only after satisfactory wound healing from the surgery;
(vi) disease-free status as documented by a complete physical examination and imaging studies prior to treatment;
(vii) patient shows ≥1% LAG3 in the tumor tissue as determined by IHC or iPET; and
(viii) patients must not have received systemic anti-cancer therapy or radiation therapy for melanoma in the previous 5 years.

26. The method of any one of claims 1 to 22, wherein the melanoma is completely resected high-risk melanoma.

27. The method of claim 26, wherein the patient is further selected as having one or more of the following criteria:
(1) ≥12 years of age;
(2) Stage IIc, III or IV (all M-stages) and histologically confirmed melanoma, completely resected <12 weeks prior to treatment;
(3) no prior systemic anti-cancer therapy or radiation therapy for melanoma in the previous 5 years;
(4) shows ≥1% LAG3 in the tumor tissue as determined by IHC or iPET;
(5) no evidence of metastatic disease; and
(6) an Eastern Cooperative Oncology Group performance status (PS) of 0 or 1 (for adult pts), Karnofsky PS>70 (pts>16 years) or Lansky PS>70 (pts<16 years).

28. The method of any one of claims 1 to 27, wherein the treatment produces a therapeutic effect selected from the group consisting of delay in melanoma growth, reduction in melanoma cell number, melanoma regression, increase in survival, partial response, and complete response.

29. The method of claim 28, wherein melanoma growth is delayed by at least 10 days as compared to an untreated subject.

30. The method of claim 28, wherein the melanoma growth is inhibited by at least 50% as compared to an untreated subject.

31. The method of claim 28, wherein the melanoma growth is inhibited by at least 20% as compared to a subject administered with either antibody as monotherapy.

32. The method of any one of claims 1 to 31 further comprising administering to the subject an additional therapeutic agent or therapy, wherein the additional therapeutic agent or therapy is selected from the group consisting of radiation, surgery, a chemotherapeutic agent, a cancer vaccine, a PD-L1 inhibitor, a CTLA-4 inhibitor, a TIM3 inhibitor, a BTLA inhibitor, a TIGIT inhibitor, a CD47 inhibitor, a CD28 agonist, a CD38 inhibitor, an indoleamine-2,3-dioxygenase (IDO) inhibitor, a vascular endothelial growth factor (VEGF) antagonist, an angiopoietin-2 (Ang2) inhibitor, a transforming growth factor beta (TGFβ) inhibitor, an epidermal growth factor receptor (EGFR) inhibitor, an antibody to a tumor-specific antigen, Bacillus Calmette-Guerin vaccine, granulocyte-macrophage colony-stimulating factor, an oncolytic virus, a cytotoxin, an interleukin 6 receptor (IL-6R) inhibitor, an interleukin 4 receptor (IL-4R) inhibitor, an IL-10 inhibitor, IL-2, IL-7, IL-21, IL-12, IL-15, an antibody-drug conjugate, a GITR agonist, a 4-1BB agonist, CD20xCD3 bispecific antibody, MUC16xCD3 bispecific antibody, and an anti-inflammatory drug.

33. The method of any one of claims 1 to 32, wherein the anti-PD-1 antibody or antigen-binding fragment thereof comprises the heavy chain complementarity determining regions (HCDR1, HCDR2 and HCDR3) of a heavy chain variable region (HCVR) and three light chain complementarity determining regions (LCDR1, LCDR2 and LCDR3) of a light chain variable region (LCVR), wherein HCDR1 comprises the amino acid sequence of SEQ ID NO: 3; HCDR2 comprises the amino acid sequence of SEQ ID NO: 4; HCDR3 comprises the amino acid sequence of SEQ ID NO: 5; LCDR1 comprises the amino acid sequence of SEQ ID NO: 6; LCDR2 comprises the amino acid sequence of SEQ ID NO: 7; and LCDR3 comprises the amino acid sequence of SEQ ID NO: 8.

34. The method of claim 33, wherein the HCVR comprises the amino acid sequence of SEQ ID NO: 1 and the LCVR comprises the amino acid sequence of SEQ ID NO: 2.

35. The method of any one of claims 1 to 34, wherein the anti-PD-1 antibody comprises a heavy chain comprising the amino acid sequence of SEQ ID NO: 9 and a light chain comprising the amino acid sequence of SEQ ID NO: 10.

36. The method of any one of claims 1 to 35, wherein the anti-LAG-3 antibody or antigen-binding fragment thereof comprises the heavy chain CDRs (HCDR1, HCDR2 and HCDR3) of a HCVR and three light chain CDRs (LCDR1, LCDR2 and LCDR3) of a LCVR, wherein HCDR1 comprises the amino acid sequence of SEQ ID NO: 13; HCDR2 comprises the amino acid sequence of SEQ ID NO: 14; HCDR3 comprises the amino acid sequence of SEQ ID NO: 15; LCDR1 comprises the amino acid sequence of SEQ ID NO: 16; LCDR2 comprises the amino acid sequence of SEQ ID NO: 17; and LCDR3 comprises the amino acid sequence of SEQ ID NO: 18.

37. The method of claim 36, wherein the HCVR comprises the amino acid sequence of SEQ ID NO: 11 and the LCVR comprises the amino acid sequence of SEQ ID NO: 12.

38. The method of any one of claims 1 to 37, wherein the anti-LAG-3 antibody comprises a heavy chain comprising the amino acid sequence of SEQ ID NO: 19 and a light chain comprising the amino acid sequence of SEQ ID NO: 20.

39. The method of any one of claims 1 to 38, wherein the inhibition is more efficacious than administration of either antibody as a monotherapy.

40. A method of treating melanoma or inhibiting the growth of melanoma comprising:
(1) selecting a patient with melanoma, wherein the patient has unresectable locally advanced melanoma, unresectable metastatic melanoma, or completely resected high-risk melanoma; and
(2) administering to the patient (a) 350 mg anti-PD-1 antibody or antigen-binding fragment thereof comprising the HCVR/LCVR amino acid sequence pair of SEQ ID NOs: 1/2; and (b) 400 mg or 1600 mg anti-LAG-3 antibody or antigen-binding fragment thereof comprising the HCVR/LCVR amino acid sequence pair of SEQ ID NOs: 11/12.

41. The method of claim 40, wherein the administering of step (2) occurs once every 3 weeks.

42. The method of claim 40, wherein the administering of step (2) occurs once every 6 weeks.

43. The method of claim 40, wherein the patient is further selected as having one or more of the following criteria:
(1) ≥12 years of age;
(2) Stage IIc, III or IV (all M-stages) and histologically confirmed melanoma, completely resected <12 weeks prior to treatment;
(3) no prior systemic anti-cancer therapy or radiation therapy for melanoma in the previous 5 years;
(4) no evidence of metastatic disease; and
(5) an Eastern Cooperative Oncology Group performance status (PS) of 0 or 1 (for adult pts), Karnofsky PS>70 (pts>16 years) or Lansky PS>70 (pts<16 years).

44. A method of treating melanoma or inhibiting the growth of melanoma comprising:
(1) selecting a patient with an unresectable locally advanced melanoma, unresectable metastatic melanoma, or completely resected high-risk melanoma, wherein the selected patient has not received prior systemic treatment for advanced disease; and
(2) administering to the patient (a) 350 mg anti-PD-1 antibody or antigen-binding fragment thereof comprising the HCVR/LCVR amino acid sequence pair of SEQ ID NOs: 1/2; and (b) 400 mg or 1600 mg anti-LAG-3 antibody or antigen-binding fragment thereof comprising the HCVR/LCVR amino acid sequence pair of SEQ ID NOs: 11/12.

45. The method of claim 44, wherein the administering of step (2) occurs once every 3 weeks.

46. The method of claim 44, wherein the administering of step (2) once every 6 weeks.

47. The method of claim 44, wherein the patient is further selected as having one or more of the following criteria:
(1) ≥12 years of age;
(2) Stage IIc, III or IV (all M-stages) and histologically confirmed melanoma, completely resected <12 weeks prior to treatment;
(3) no prior systemic anti-cancer therapy or radiation therapy for melanoma in the previous 5 years;
(4) no evidence of metastatic disease on staging investigations; and
(5) an Eastern Cooperative Oncology Group performance status (PS) of 0 or 1 (for adult pts), Karnofsky PS>70 (pts>16 years) or Lansky PS>70 (pts<16 years).

48. A method of treating melanoma or inhibiting the growth of melanoma comprising:
(1) selecting a patient with a melanoma; and
(2) administering to the patient (a) 400 mg or 1600 mg anti-LAG-3 antibody or antigen-binding fragment thereof comprising the HCVR/LCVR amino acid sequence pair of SEQ ID NOs: 11/12 in combination with (a), (b) 350 mg anti-PD-1 antibody or antigen-binding fragment thereof comprising the HCVR/LCVR amino acid sequence pair of SEQ ID NOs: 1/2.

49. The method of claim 48, wherein the step of administering occurs once every 3 weeks.

50. The method of claim 48, wherein the step of administering occurs once every 6 weeks.

51. A method of treating melanoma or inhibiting the growth of melanoma comprising:
(a) selecting a patient with melanoma wherein the patient has completed surgery to treat the melanoma; and
(b) administering to the patient:
(1) an initial loading dose comprising an anti-PD-1 antibody or antigen-binding fragment thereof comprising the HCVR/LCVR amino acid sequence pair of SEQ ID NOs: 1/2; and an anti-LAG-3 antibody or antigen-binding fragment thereof comprising the HCVR/LCVR amino acid sequence pair of SEQ ID NOs: 11/12; and
(2) one or more secondary doses, wherein the one or more secondary doses occur one to four weeks after the immediately preceding dose.

52. The method of claim 51, wherein the patient shows 1% of more LAG3 in the melanoma tissue.

53. The method of claim 51, wherein the patient is diagnosed with Stage IV melanoma.

54. The method of claim 51, wherein the patient is further selected as having one or more of the following criteria:
(1) ≥12 years of age;
(2) Stage IIc, III or IV (all M-stages) and histologically confirmed melanoma, completely resected <12 weeks prior to treatment;
(3) no prior systemic anti-cancer therapy or radiation therapy for melanoma in the previous 5 years;
(4) shows ≥1% LAG3 in the tumor tissue as determined by IHC or iPET;
(5) no evidence of metastatic disease; and
(6) an Eastern Cooperative Oncology Group performance status (PS) of 0 or 1 (for adult pts), Karnofsky PS>70 (pts>16 years) or Lansky PS>70 (pts<16 years).

55. The method of claim 51, further comprising administering to a patient in need thereof:
(3) one or more tertiary doses, wherein the one or more tertiary doses occur three to twelve weeks after the immediately preceding dose.

56. The method of claim 51, wherein the one or more secondary doses occur three weeks after the immediately preceding dose.

54. The method of claim 55, wherein the one or more tertiary doses occur three weeks or six weeks after the immediately preceding dose.

58. The method of claim 51, wherein the initial loading dose comprises (a) 500 mg to 1500 mg anti-PD-1 antibody or antigen-binding fragment thereof and (b) 50 mg to 8000 mg anti-LAG-3 antibody or antigen-binding fragment thereof.

59. The method of claim 51, wherein the one or more secondary doses comprise: (a) 350 mg anti-PD-1 antibody or antigen-binding fragment thereof and (b) 400 mg, 800 mg, 1000 mg, 1400 mg, 1600 mg or 2000 mg anti-LAG-3 antibody or antigen-binding fragment thereof.

60. The method of claim 55, wherein the one or more tertiary doses comprise: (a) 350 mg anti-PD-1 antibody or antigen-binding fragment thereof and (b) 400 mg, 800 mg, 1000 mg, 1400 mg, 1600 mg or 2000 mg anti-LAG-3 antibody or antigen-binding fragment thereof.


SEQUENCE LISTING (20 sequences)

SEQ ID NO: 1 - protein, 117 aa, synthetic construct [sequence omitted]

SEQ ID NO: 2 - protein, 107 aa, synthetic construct [sequence omitted]

SEQ ID NO: 3 - protein, 8 aa, synthetic construct
  REGION 1..8: note = Synthetic
GFTFSNFG

SEQ ID NO: 4 - protein, 8 aa, synthetic construct
  REGION 1..8: note = Synthetic
ISGGGRDT

SEQ ID NO: 5 - protein, 10 aa, synthetic construct
  REGION 1..10: note = Synthetic
VKWGNIYFDY

SEQ ID NO: 6 - protein, 6 aa, synthetic construct
  REGION 1..6: note = Synthetic
LSINTF

SEQ ID NO: 7 - skipped

SEQ ID NO: 8 - protein, 9 aa, synthetic construct
  REGION 1..9: note = Synthetic
QQSSNTPFT

SEQ ID NO: 9 - protein, 444 aa, synthetic construct
  REGION 1..444: note = Synthetic
EVQLLESGGVLVQPGGSLRLSCAASGFTFSNFGMTWVRQAPGKGLEWVSGISGGGRDTYFADSVKGRFTISRDNSKNTLYLQMNSLKGEDTAVYYCVKWGNIYFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 10 - protein, 214 aa, synthetic construct
  REGION 1..214: note = Synthetic
DIQMTQSPSSLSASVGDSITITCRASLSINTFLNWYQQKPGKAPNLLIYAASSLHGGVPSRFSGSGSGTDFTLTIRTLQPEDFATYYCQQSSNTPFTFGPGTVVDFRRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 11 - protein, 123 aa, synthetic construct
  REGION 1..123: note = Synthetic
QVQLVESGGGVVQPGRSLRLSCVASGFTFSSYGMHWVRQAPGKGLEWVAIIWYDGSNKYYADSVKGRFTISRDNSKNTQYLQMNSLRAEDTAVYYCASVATSGDFDYYGMDVWGQGTTVTVSS

SEQ ID NO: 12 - protein, 107 aa, synthetic construct
  REGION 1..107: note = Synthetic
EIVLTQSPATLSLSPGERTTLSCRASQRISTYLAWYQQKPGQAPRLLIYDASKRATGIPARFSGSGSGTGFTLTISSLEPEDFAVYYCQQRSNWPLTFGGGTKVEIK

SEQ ID NO: 13 - protein, 8 aa, synthetic construct
  REGION 1..8: note = Synthetic
GFTFSSYG

SEQ ID NO: 14 - protein, 8 aa, synthetic construct
  REGION 1..8: note = Synthetic
IWYDGSNK

SEQ ID NO: 15 - protein, 16 aa, synthetic construct
  REGION 1..16: note = Synthetic
ASVATSGDFDYYGMDV

SEQ ID NO: 16 - protein, 6 aa, synthetic construct
  REGION 1..6: note = Synthetic
QRISTY

SEQ ID NO: 17 - skipped

SEQ ID NO: 18 - protein, 9 aa, synthetic construct
  REGION 1..9: note = Synthetic
QQRSNWPLT

SEQ ID NO: 19 - protein, 449 aa, synthetic construct
  REGION 1..449: note = Synthetic
QVQLVESGGGVVQPGRSLRLSCVASGFTFSSYGMHWVRQAPGKGLEWVAIIWYDGSNKYYADSVKGRFTISRDNSKNTQYLQMNSLRAEDTAVYYCASVATSGDFDYYGMDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 20 - protein, 214 aa, synthetic construct
  REGION 1..214: note = Synthetic
EIVLTQSPATLSLSPGERTTLSCRASQRISTYLAWYQQKPGQAPRLLIYDASKRATGIPARFSGSGSGTGFTLTISSLEPEDFAVYYCQQRSNWPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC